(19) United States
(12) Patent Application Publication
(10) Pub. No.: US 2025/0235517 A1
(43) Pub. Date: Jul. 24, 2025

(54) TUMOR-ASSOCIATED ANTIGENS IN BRAIN TUMORS

(71) Applicant: IOGENETICS, LLC, Sun Prairie, WI (US)

(72) Inventors: Jane Homan, Sun Prairie, WI (US); Robert D. Bremel, Sun Prairie, WI (US)

(21) Appl. No.: 18/852,707

(22) PCT Filed: Mar. 27, 2023
(86) PCT No.: PCT/US2023/064988

§ 371 Date: Sep. 30, 2024

Related U.S. Application Data
(60) Provisional application No. 63/325,220, filed on Mar. 30, 2022.

(51) Int. Cl. A61K 39/00; A61K 40/11; A61K 40/42; A61P 35/00; C12N 5/0783; C12Q 1/6881; G01N 33/574

(52) U.S. Cl. CPC A61K 39/0011; A61K 40/11; A61K 40/42; A61P 35/00; C12N 5/0636; C12Q 1/6881; G01N 33/57407; C12N 2510/00; C12Q 2600/156

(57) ABSTRACT

The present invention relates to the identification and use of tumor epitopes from subjects with brain cancer, and particularly to epitopes from brevican, neurocan, versican, and aggrecan and their use in formulating cancer vaccines for treatment of tumor patients. In some preferred embodiments the methods provide a means of identifying T cell epitopes in proteins upregulated in brain tumors and the selection of those peptides which can stimulate T cell responses in individual subjects with a particular combination of HLA alleles. It further identifies the T cell exposed motifs comprised in T cell epitopes and enables the design of peptides with alternative amino acids in positions other than the T cell exposed motifs. In preferred embodiments, the MHC I and MHC II alleles of the affected subject are determined, and peptides are selected which bind to their MHC molecules with a desired affinity to elicit stimulation.

### CROSS REFERENCE TO RELATED APPLICATION

This application claims priority to U.S. provisional patent application Ser. No. 63/325,220 filed Mar. 30, 2022, which is incorporated herein by reference in its entirety.

### SEQUENCE LISTING

The text of the computer readable sequence listing filed herewith, titled “40620-601_SequenceListing”, created Mar. 23, 2023, having a file size of 283,630 bytes, is hereby incorporated by reference in its entirety.

### FIELD OF THE INVENTION

The present invention relates to the identification and use of tumor epitopes from subjects with brain cancer, and particularly to epitopes from brevican, neurocan, versican, and aggrecan and their use in formulating cancer vaccines for treatment of tumor patients.

### BACKGROUND OF THE INVENTION

Tumors arise from genetic mutations in certain genes which lead to loss or gain of function and which have evaded immune surveillance. Two approaches have been advanced as a means of stimulating an immune response which can arrest tumor progression. In both cases the goal is to direct an immune response to tumor epitopes without leading to collateral damage to normal tissue or adverse effects arising from autoimmunity. One approach targets neoantigens which are novel epitopes that result from a mutation that causes a change in a protein sequence expressed by the tumor. A second, older approach, targets tumor associated antigens that are epitopes in proteins that are found in proximity to and upregulated in tumors. Tumor associated antigens are also found in normal tissues and thus it is critical to focus such an approach tightly on those proteins the upregulation and location of which is highly specific to the tumor.

Gliomas are the most common malignant brain tumor in the United States, with an average of 6 cases per 100,000. Glioblastoma is the most common form of glioma and has a 5 year survival rate of only 5% [1]. Interventions which can improve the outcome of such cancers are thus urgently needed.

Brevican is a protein that is expressed only in glial cells and which is highly upregulated in gliomas, including low and high grade gliomas, glioblastomas, astrocytomas, ependymomas, and oligodendrocytomas. Expression of brevican is correlated to progression and invasion of gliomas. Experimentally, knock down of brevican can limit tumor progression. Brevican has thus been identified as a target for immunotherapy. The present invention provides methods for selection and design of peptides which can elicit T cell immunity to brevican, using both natural peptides and modified peptides designed to bind optimally to the MHC alleles of an individual subject affected by a brain tumor. The T cells resulting from such stimulation may also be the source of T cell receptor sequences for recombinant engineering into additional T cell clones for administration to the patient. The T cell clones stimulated by immunization with the selected peptides may also be harvested and expanded for re-administration in adoptive cell therapy. The present invention also identifies B cell epitopes and peptides that may be applied to elicit an antibody response and result in antibody dependent cell mediated cytotoxicity directed to brevican-bearing cells. The antibody variable regions and subcomponent antigen binding molecules derived therefrom may also be utilized in the engineering of CAR-T cells directed to gliomas.

The invention also provides a vaccination regimen to administer the selected peptides to the subject affected with a brain tumor. As manufacturability of peptides is a consideration, it also addresses the selection and design of peptides which have desired characteristics for manufacturing and formulation. The invention further provides multiple modes of delivery of a brevican targeting vaccine.

### SUMMARY OF THE INVENTION

The present invention provides methods for targeting immunotherapy for subjects affected by brain tumors. In particular, it addresses the selection and design of epitopes in brevican to elicit T cell and B cell responses in such subjects.

In some preferred embodiments the methods provide a means of identifying T cell epitopes in proteins upregulated in brain tumors and the selection of those peptides which can stimulate T cell responses in individual subjects with a particular combination of HLA alleles. It further identifies the T cell exposed motifs comprised in T cell epitopes and enables the design of peptides with alternative amino acids in positions other than the T cell exposed motifs. In preferred embodiments, the MHC I and MHC II alleles of the affected subject are determined, and peptides are selected which bind to their MHC molecules with a desired affinity to elicit stimulation. In other preferred embodiments, the substitution of amino acids in those positions in the peptide that do not lie within the T cell exposed motif allows the design of peptides with an increased MHC binding affinity for particular HLA alleles, thereby allowing stimulation of T cell clones cognate for epitopes which have sub-dominant binding for the subject's particular HLA alleles. In both instances the methods provide a means to identify upregulated proteins that are specific to the tumor, and to select or design an array of one or more peptides that can be administered to induce a T cell response directed to the upregulated protein. The methods further provide for an embodiment in which the selected array of peptides are encoded in a nucleic acid sequence for administration.

In preferred embodiments the invention enables immune responses to be directed to tumors of glial cell origin. These may be low or high grade gliomas, glioblastomas, astrocytomas, ependymomas and oligodendrocytomas. In the embodiments described the immune response is targeted to proteins in the extracellular matrix, including but not limited to brevican, neurocan, versican, and aggrecan. In the most preferred embodiments, the immune response is directed to brevican. Sequences are provided herein that identify epitope peptides of interest in brevican suitable to induce an immune response.

In some embodiments the upregulation of brevican is verified by conducting sequencing of tumor biopsy. In yet other embodiments, given the near-uniformity of brevican upregulation on glioblastoma and higher grade gliomas, immunization may be initiated on clinical diagnosis without awaiting a biopsy and sequencing, provided that the subject's H1LA are determined.

In some embodiments the T cell epitopes comprise peptides which bind to an MHC I molecule, whereas in other embodiments the T cell epitopes comprise peptides which bind to an MHC II molecule. Accordingly, preferred embodiments include peptides of 8-10 amino acids to bind MHC I alleles and peptides of 11-22 amino acids to bind MHC II alleles. In most preferred embodiments a selected array of peptides includes one or more peptides binding an MHC I molecule and one or more peptides binding an MHC II molecule.

The invention provides peptides, or the nucleic acids which encode them, with a binding affinity which is suitable to provide for T cell stimulation and not lead to exhaustion or anergy of the T cells. A desired binding affinity is found when such binding affinity is less than 200 nanomolar; in yet more preferred instances the binding affinity may be less than 100 nanomolar or less than 50 nanomolar or 20 nanomolar. However, the methods also provide a means to avoid selection of peptides which are excessively highly bound. The methods also demonstrate that certain peptides may have such a high binding affinity for a particular allele that they have a potential of leading to T cell exhaustion and should be avoided. Such peptides are found in the top approximately 0.61% of MHC binding peptides in the protein as a whole.

Full length brevican is cleaved by metalloproteinase ADAMTS4 prior to Ser401 to release an N terminal polypeptide. In one embodiment the invention provides peptide sequences in the N terminal cleavage product of brevican that bind one or more MHC I alleles with a desired affinity. In a further embodiment it provides peptide sequences in the N terminal cleavage product of brevican that bind one or more MHC II alleles. Selection from this list of peptides is made by examining the binding affinity for the HLA alleles carried by the patient. Certain peptides are suitable for one patient but not another, based on their HLA alleles. In a further embodiment, the T cell exposed motifs which lie within the epitopes of the N terminal cleavage product of brevican are identified. Then, in a further preferred embodiment, exemplar sequences are provided for peptides which have been personalized for a particular subject by substitution of the amino acids not located in the T cell exposed motifs, to provide optimal binding for that particular subject's HLA alleles. It will be appreciated that this is an example of the design of a personalized peptide array from brevican, and that a similar array could be generated by the same methods to provide binding of sub-dominant epitopes for a subject of a different HLA allele composition. Thus, this example is not considered limiting. Indeed, in some instances the subject does not carry an A0201 allele or a A2402 allele, and the embodiments demonstrate that the sequences provided are suitable for individuals carrying other alleles.

As a goal of tumor immunotherapy is to target tumor tissue but leave normal tissue unharmed, the present invention, in one embodiment, targets those proteins which are upregulated, such that the normalized mRNA transcript expression of the protein is in the top 15% of the tumor proteome. In yet more highly preferred embodiments the normalized mRNA transcript expression of the protein is in the top 2.25% of the tumor proteome. A further goal of tumor immunotherapy is to target the tumor but to avoid collateral damage to the subject through autoimmunity. Thus, an initial selection of peptides is compared to a precomputed database of epitopes within the human proteome in its entirety. Any peptide carrying a T cell exposed motif which, when in the context of a peptide in a different and critical protein in the proteome the targeting of which could have adverse effects, is predicted to be bound to the particular subject's MHC with high affinity is omitted from the selection.

In another embodiment the present invention provides a means of targeting B cell epitopes in proteins upregulated in brain tumors, in order to stimulate the production of antibodies. In preferred embodiments the upregulated protein is brevican. The invention provides sequences that comprise linear B cell epitopes in the N terminal cleavage product of brevican and also sequences that comprise B cell epitopes in the C terminal cleavage product. These epitopes may be administered to a subject as a peptide or as a nucleic acid encoding a peptide. While the N terminal 400 amino acid cleavage product of brevican is linked to the invasiveness of gliomas, the C terminal portion may be retained by the glial cell expressing brevican. As the minimal motif which can form a linear B cell epitope and can bind within the complementarity determining region of an immunoglobulin is 5 amino acids, another embodiment provides the B cell epitope core pentamers in the B cell epitopes of brevican. In addition to direct administration of B cell epitope peptides, or their encoding nucleic acids to the affected subject, in a further embodiment the invention provides for production of an antibody by immunization of a subject with the designated B cell epitope peptides, and the production of antigen binding molecules derived from such antibodies. Such antigen binding molecules include, but are not limited to, single chain variable region fragments (scFV). In some embodiments the subject thus immunized is a human subject, but in other embodiments the subject may be a non-human animal.

Methods are also provided by this invention for selecting and designing peptides from the proteins of interest, from among those selected based on their role as epitopes, in order to facilitate their formulation and delivery. In some embodiments this is achieved by the selection of peptides based on their characteristics of solubility, stability, and to reduce aggregation. In preferred embodiments peptides are selected based on their polarity, and in particular where the average of the first principal components of the amino acids in the peptides is less than or equal to 1 or 2. Another index considered is the octanol:water log P, which in preferred embodiments is less than or equal to −2.

Certain amino acids are prone to affect solubility. In preferred embodiments the amino acids arginine, lysine, aspartic acid, and glutamic acid are excluded from the amino acids not located in the T cell exposed motif Other amino acids are more likely to affect stability; these are methionine, tryptophan, histidine, cysteine and tyrosine. In some preferred embodiments they are excluded from the amino acids not located in the T cell exposed motif Similar considerations apply to asparagine and glutamine.

In preferred embodiments the peptides selected for T or B cell stimulation are selected to have a molecular weight less than 4000 daltons, or in preferred embodiments less than 1500 daltons.

The invention then provides methods for a vaccination regimen for administration to a subject. The vaccination comprises the selected one or more peptides, which may comprise T cell stimulating epitopes or B cell stimulating epitopes. The subject may be affected with a brain tumor which has been confirmed by biopsy, or be clinically diagnosed as having a probable brain tumor. The vaccination regimen may comprise peptides that bind MHC I or MHC II alleles. In yet more preferred embodiments the regimen may comprise one or more peptides from both MHC I and MHC II binding groups. In yet other embodiments the vaccination also comprises the administration of one or more B cell epitope peptides. In some embodiments the vaccination may be accompanied by, or followed by, an immunotherapy intervention, such as but not limited to, the administration of a check point inhibitor.

Various routes of administration of the vaccination regimen are feasible and this invention provides for parenteral administration, including but not limited to, intradermal and subcutaneous routes, as well as non-parenteral routes, including but not limited to, intranasal, pulmonary inhalation, rectal, and oral routes. Said oral routes include buccal, pharyngeal and sublingual routes, as well as delivery to the gastrointestinal tract. Further embodiments provide for formulations appropriate to each route, including but not limited to, coated tablets and capsules, including enteric coated capsules. Additional embodiments are for the delivery of the vaccinal peptides as lipid drug delivery systems, which may include lipid nanoparticles, emulsions, self-emulsifying drug delivery systems, nanocapsules, or liposomes. In alternative embodiments delivery is in a particulate form. In further embodiments the vaccine described herein may be delivered by other means including, but not limited to, a nanoparticle system, a hydrogel system, a mucoadhesive patch, or a microneedle. A particular preferred embodiment is a microneedle patch for delivery to the dermis, or delivery by a multineedle delivery device.

The vaccination regimens for both T cell and B cell stimulation may, in preferred embodiments, be delivered with an adjuvant. Many adjuvants are well known to the art and non-limiting examples are described herein. In some particular instances the adjuvant may be administered prior to the vaccine. Peptides applied in vaccinations described herein may be delivered with a pharmaceutical excipient or alternatively may be lyophilized.

In another embodiment, rather than administer the peptides or their encoding nucleic acids directly to the affected subject, dendritic cells drawn from the subject may be contacted with the selected vaccinal peptides and cultured in vitro, before then further contacting them with T cells in vitro or by reinfusion into the subject of origin.

Additional embodiments enabled by the present invention provide for the adoptive cell transfer of T cells or modified T cells. In one embodiment, following administration of T cell stimulating peptides to the subject of interest, T cells are harvested from the subject. Those T cells which are cognate for the epitopes comprised in the selected peptides, when these are presented bound in the MHC molecules of the subject, are multiplied in vitro and transferred back into the subject in larger numbers. In a further preferred embodiment once the epitope-cognate T cells are identified in vitro, the T cell receptor sequences are determined and these sequences are then engineered into additional T cells, which may be of allogenic or autologous origin. The engineered T cells are then transferred into the subject. A further embodiment involving adoptive cell transfer, provided herein, is the engineering of CAR-T (chimeric antigen receptor bearing cells) in which antigen binding molecules derived from antibodies elicited by administration of the B cell epitopes described herein are engineered on to T cells. Said T cells may be of autologous or allogenic origin. The CAR-T cells are then administered to the subject who has been diagnosed as having a brain tumor.

### DESCRIPTION OF FIGURES

FIG. 1: Upregulation of expression of brevican in different cancers. Y axis shows RNA expression in standard deviation units (Z-scale) of FPKM (fragments per kilobase million). Cancer types are identified on the X axis by their TCGA acronyms. Top tier shows the upregulation of the long isoform 1 (Q96GW7). Lower tier shows expression of the 671 amino acid isoform 2.

FIG. 2: Epitopes in Brevican Isoform 1 (Q96GW7). Overview of MHC binding, B cell epitopes and topology. The X axis indicates the index position of sequential peptides with single amino acid displacement. The Y axis indicates predicted binding affinity of each peptide in standard deviation units for the protein. The red line shows the permuted average predicted MHC-IA and B (62 alleles) binding affinity of sequential 9-mer peptides with single amino acid displacement. The blue line shows the permuted average predicted MHC-II DRB allele (24 most common human alleles) binding affinity of sequential 15-mer peptides. Orange lines show the predicted probability of B-cell receptor binding for an amino acid centered in each sequential 9-mer peptide. Low numbers for MHC data represent high binding affinity, whereas low numbers equate to high B cell receptor contact probability. Ribbons (red: MHC-I, blue: MHC-1I) indicate the 10% highest predicted MHC affinity binding. Orange ribbons indicate the top 25% predicted probability B-cell binding. Horizontal dotted lines demarcate the top 5% of binding affinity for the protein (red MHC I, blue MHC II). The black line between amino acid positions 400-401 indicates the cleavage site.

FIG. 3: Epitopes in Brevican Isoform 2 (Q96GW7-2). Legend as for FIG. 2.

FIG. 4: MHC allele specific differences in binding of exemplar MHC I alleles (5 MHC I A and 5 MHC IB) at regions of brevican with highest predicted binding. Y axis shows binding in standard deviation units relative to the mean of all peptides in brevican isoform 1 (negative values have higher affinity). X axis shows index amino acid position of 9 mer peptides. Legend indicated allele symbols.

FIG. 5: Shows the distribution of binding affinity of all 9-mer peptides in brevican isoform 1 for A0201 and A2402 and highlights those peptides which are in the top approximately 0.61% binding affinity for the two alleles and considered to bind excessively to the corresponding allele. The X axis of the distribution is provided in standard deviation units. Note that in the case of A0201 one of these peptides would be discarded as it lies in the signal peptide.

### DEFINITIONS

As used herein, the term “genome” refers to the genetic material (e.g., chromosomes) of an organism or a host cell.

As used herein, the term “proteome” refers to the entire set of proteins expressed by a genome, cell, tissue or organism. A “partial proteome” refers to a subset the entire set of proteins expressed by a genome, cell, tissue or organism. Examples of “partial proteomes” include, but are not limited to, transmembrane proteins, secreted proteins, and proteins with a membrane motif. Human proteome refers to all the proteins comprised in a human being. Multiple such sets of proteins have been sequenced and are accessible at the InterPro international repository (world wide web at ebi.ac.uklinterpro). Human proteome is also understood to include those proteins and antigens thereof which may be over-expressed in certain pathologies, or expressed in a different isoforms in certain pathologies. Hence, as used herein, tumor associated antigens are considered part of the human proteome. “Proteome” may also be used to describe a large compilation or collection of proteins, such as all the proteins in an immunoglobulin collection or a T cell receptor repertoire, or the proteins which comprise a collection such as the allergome, such that the collection is a proteome which may be subject to analysis. All the proteins in a bacteria or other microorganism are considered its proteome.

As used herein, the terms “protein,” “polypeptide,” and “peptide” refer to a molecule comprising amino acids joined via peptide bonds. In general “peptide” is used to refer to a sequence of 40 or less amino acids and “polypeptide” is used to refer to a sequence of greater than 40 amino acids.

As used herein, the term, “synthetic polypeptide,” “synthetic peptide” and “synthetic protein” refer to peptides, polypeptides, and proteins that are produced by a recombinant process (i.e., expression of exogenous nucleic acid encoding the peptide, polypeptide or protein in an organism, host cell, or cell-free system) or by chemical synthesis.

As used herein, the term “protein of interest” refers to a protein encoded by a nucleic acid of interest. It may be applied to any protein to which further analysis is applied or the properties of which are tested or examined. Similarly, as used herein, “target protein” may be used to describe a protein of interest that is subject to further analysis.

As used herein the term “amino acid of interest” refers to an amino acid which sets the protein apart from other sequences of the same protein, for instance by being the product of a mutation, indel, splice or fusion event, or the amino acid attracts attention as it is a salient feature in a particular T cell epitope, or the amino acid is associated with a function such as binding to a particular receptor or the basis for post translational modifications.

A “target peptide” as used herein is one to which it is desired to direct an immune response.

As used herein “peptidase” refers to an enzyme which cleaves a protein or peptide. The term peptidase may be used interchangeably with protease, proteinases, oligopeptidases, and proteolytic enzymes. Peptidases may be endopeptidases (endoproteases), or exopeptidases (exoproteases). The the term peptidase would also include the proteasome which is a complex organelle containing different subunits each having a different type of characteristic scissile bond cleavage specificity. Similarly the term peptidase inhibitor may be used interchangeably with protease inhibitor or inhibitor of any of the other alternate terms for peptidase.

As used herein, the term “immunogen” refers to a molecule which stimulates a response from the adaptive immune system, which may include responses drawn from the group comprising an antibody response, a cytotoxic T cell response, a T helper response, and a T cell memory. An immunogen may stimulate an upregulation of the immune response with a resultant inflammatory response, or may result in down regulation or immunosuppression. Thus the T-cell response may be a T regulatory response. An immunogen also may stimulate a B-cell response and lead to an increase in antibody titer. Another term used herein to describe a molecule or combination of molecules which stimulate an immune response is “antigen”.

As used herein, the term “native” (or wild type) when used in reference to a protein refers to proteins encoded by the genome of a cell, tissue, or organism, other than one manipulated to produce synthetic proteins.

As used herein the term “epitope” refers to a peptide sequence which elicits an immune response, from either T cells or B cells or antibody As used herein, the term “B-cell epitope” refers to a polypeptide sequence that is recognized and bound by a B-cell receptor. A B-cell epitope may be a linear peptide or may comprise several discontinuous sequences which together are folded to form a structural epitope. Such component sequences which together make up a B-cell epitope are referred to herein as B-cell epitope sequences. Hence, a B-cell epitope may comprise one or more B-cell epitope sequences. Hence, a B cell epitope may comprise one or more B-cell epitope sequences. A linear B-cell epitope may comprise as few as 2-4 amino acids or more amino acids. “B cell core peptides” or “core pentamer” when used herein refers to the central 5 amino acid peptide in a predicted B cell epitope sequence. Said B cell epitope may be evaluated by predicting the binding of across a series of 9-mer windows, the core pentamer then is the central pentamer of the 9-mer window As used herein, the term “predicted B-cell epitope” refers to a polypeptide sequence that is predicted to bind to a B-cell receptor by a computer program, for example, as described in PCT US2011/029192, PCT US2012/055038, US2014/014523, PCT US2015/039969, PCT US2020/037206, U.S. Pat. Nos. 10,706,955 and 10,755,801 each of which is incorporated herein by reference in its entirety, and in addition by Bepipred (Larsen, et al., Immunome Research 2:2, 2006.) and others as referenced by Larsen et al (ibid) (Hopp T et al PNAS 78:3824-3828, 1981; Parker J et al, Biochem. 25:5425-5432, 1986). A predicted B-cell epitope may refer to the identification of B-cell epitope sequences forming part of a structural B-cell epitope or to a complete B-cell epitope.

As used herein, the term “T-cell epitope” refers to a polypeptide sequence which when bound to a major histocompatibility protein molecule provides a configuration recognized by a T-cell receptor. Typically, T-cell epitopes are presented bound to an MHC molecule on the surface of an antigen-presenting cell.

As used herein, the term “predicted T-cell epitope” refers to a polypeptide sequence that is predicted to bind to a major histocompatibility protein molecule by the neural network algorithms described herein, by other computerized methods, or as determined experimentally.

As used herein, the term “major histocompatibility complex (MHC)” refers to the MHC Class I and MHC Class II genes and the proteins encoded thereby. Molecules of the MHC bind small peptides and present them on the surface of cells for recognition by T-cell receptor-bearing T-cells. The MHC is both polygenic (there are several MHC class I and MHC class II genes) and polyallelic or polymorphic (there are multiple alleles of each gene). The terms MHC-I, MHC-II, MHC-1 and MHC-2 are variously used herein to indicate these classes of molecules. Included are both classical and nonclassical MHC molecules. An MHC molecule is made up of multiple chains (alpha and beta chains) which associate to form a molecule. The MHC molecule contains a cleft or groove which forms a binding site for peptides. Peptides bound in the cleft or groove may then be presented to T-cell receptors. The term “MHC binding region” refers to the groove region of the MHC molecule where peptide binding occurs.

As used herein, an “MHC II binding groove” refers to the structure of an MHC molecule that binds to a peptide. The peptide that binds to the MHC II binding groove may be from about 11 amino acids to about 23 amino acids in length, but typically comprises a 15-mer. The amino acid positions in the peptide that binds to the groove are numbered based on a central core of 9 amino acids numbered 1-9, and positions outside the 9 amino acid core numbered as negative (N terminal) or positive (C terminal). Hence, in a 15mer the amino acid binding positions are numbered from −3 to +3 or as follows: −3, −2, −1, 1, 2, 3, 4, 5, 6, 7, 8, 9, +1, +2, +3.

As used herein, the term “haplotype” refers to the HLA alleles found on one chromosome and the proteins encoded thereby. Haplotype may also refer to the allele present at any one locus within the MHC. Each class of MHC-Is represented by several loci: e.g., HLA-A (Human Leukocyte Antigen-A), HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-, HLA-DQA1, HLA-DQB1, HLA-DPA1, HLA-DPB1, HLA-DMA, HLA-DMB, HLA-DOA, and HLA-DOB for class II. The terms “HLA allele” and “MHC allele” are used interchangeably herein. HLA alleles are listed at hla.alleles.org/nomenclature/naming.html, which is incorporated herein by reference.

The MHCs exhibit extreme polymorphism: within the human population there are, at each genetic locus, a great number of haplotypes comprising distinct alleles—the IMGT/HLA database release (January 2020) lists 25,000 alleles (MHC I plus MHC II), many of which are represented at high frequency (>1%). MHC alleles may differ by as many as 30-aa substitutions. Different polymorphic MHC alleles, of both class I and class II, have different peptide specificities: each allele encodes proteins that bind peptides exhibiting particular sequence patterns.

The naming of new HLA genes and allele sequences and their quality control is the responsibility of the WHO Nomenclature Committee for Factors of the HLA System, which first met in 1968, and laid down the criteria for successive meetings. This committee meets regularly to discuss issues of nomenclature and has published 19 major reports documenting firstly the HLA antigens and more recently the genes and alleles. The standardization of HLA antigenic specifications has been controlled by the exchange of typing reagents and cells in the International Histocompatibility Workshops. The IMGT/HLA Database collects both new and confirmatory sequences, which are then expertly analyzed and curated before been named by the Nomenclature Committee. The resulting sequences are then included in the tools and files made available from both the IMGT/HLA Database and at hla.alleles.org.

Each HLA allele name has a unique number corresponding to up to four sets of digits separated by colons. See e.g., hla.alleles.org/nomenclature/naming.html which provides a description of standard HLA nomenclature and Marsh et al., Nomenclature for Factors of the HLA System, 2010 Tissue Antigens 2010 75:291-455. HLA-DRB1*13:01 and HLA-DRB1*13:01:01:02 are examples of standard HLA nomenclature. The length of the allele designation is dependent on the sequence of the allele and that of its nearest relative. All alleles receive at least a four digit name, which corresponds to the first two sets of digits, longer names are only assigned when necessary.

The digits before the first colon describe the type, which often corresponds to the serological antigen carried by an allele, The next set of digits are used to list the subtypes, numbers being assigned in the order in which DNA sequences have been determined. Alleles whose numbers differ in the two sets of digits must differ in one or more nucleotide substitutions that change the amino acid sequence of the encoded protein. Alleles that differ only by synonymous nucleotide substitutions (also called silent or non-coding substitutions) within the coding sequence are distinguished by the use of the third set of digits. Alleles that only differ by sequence polymorphisms in the introns or in the 5′ or 3′ untranslated regions that flank the exons and introns are distinguished by the use of the fourth set of digits. In addition to the unique allele number there are additional optional suffixes that may be added to an allele to indicate its expression status. Alleles that have been shown not to be expressed, ‘Null’ alleles have been given the suffix ‘N’. Those alleles which have been shown to be alternatively expressed may have the suffix ‘L’, ‘S’, ‘C’, ‘A’ or ‘Q’. The suffix ‘L’ is used to indicate an allele which has been shown to have ‘Low’ cell surface expression when compared to normal levels. The ‘S’ suffix is used to denote an allele specifying a protein which is expressed as a soluble ‘Secreted’ molecule but is not present on the cell surface. A ‘C’ suffix to indicate an allele product which is present in the ‘Cytoplasm’ but not on the cell surface. An ‘A’ suffix to indicate ‘Aberrant’ expression where 30 there is some doubt as to whether a protein is expressed. A ‘Q’ suffix when the expression of an allele is ‘Questionable’ given that the mutation seen in the allele has previously been shown to affect normal expression levels.

In some instances, the HLA designations used herein may differ from the standard HLA nomenclature just described due to limitations in entering characters in the databases described herein. As an example, DRB1_0104, DRB1*0104, and DRB1-0104 are equivalent to the standard nomenclature of DRB1*01:04. In most instances, the asterisk is replaced with an underscore or dash and the semicolon between the two digit sets is omitted.

As used herein, the term “polypeptide sequence that binds to at least one major histocompatibility complex (MHC) binding region” refers to a polypeptide sequence that is recognized and bound by one or more particular MHC binding regions as predicted by the neural network algorithms described herein or as determined experimentally.

As used herein the terms “canonical” and “non-canonical” are used to refer to the orientation of an amino acid sequence. Canonical refers to an amino acid sequence presented or read in the N terminal to C terminal order; non-canonical is used to describe an amino acid sequence presented in the inverted or C terminal to N terminal order. “Canonical” is also used to designate the dominant sequence of a protein for which many isoforms exist. The canonical protein is thus typically that in the Reference sequence designated by uniport.org.

As used herein, the term “allergen” refers to an antigenic substance capable of producing immediate hypersensitivity and includes both synthetic as well as natural immunostimulant peptides and proteins. Allergen includes but is not limited to any protein or peptide catalogued in the Structural Database of Allergenic Proteins database fermi.utmb.edu/SDAP/index.html As used herein, the term “transmembrane protein” refers to proteins that span a biological membrane. There are two basic types of transmembrane proteins. Alpha-helical proteins are present in the inner membranes of bacterial cells or the plasma membrane of eukaryotes, and sometimes in the outer membranes. Beta-barrel proteins are found only in outer membranes of Gram-negative bacteria, cell wall of Gram-positive bacteria, and outer membranes of mitochondria and chloroplasts.

As used herein, the term “affinity” refers to a measure of the strength of binding between two members of a binding pair, for example, an antibody and an epitope and an epitope and a MHC-I or II haplotype. K_{d} is the dissociation constant and has units of molarity. The affinity constant is the inverse of the dissociation constant. An affinity constant is sometimes used as a generic term to describe this chemical entity. It is a direct measure of the energy of binding. The natural logarithm of K is linearly related to the Gibbs free energy of binding through the equation ΔG₀=-RT LN(K) where R=gas constant and temperature is in degrees Kelvin. Affinity may be determined experimentally, for example by surface plasmon resonance (SPR) using commercially available Biacore SPR units (GE Healthcare) or in silico by methods such as those described herein in detail. Affinity may also be expressed as the ic50 or inhibitory concentration 50, that concentration at which 50% of the peptide is displaced. Likewise ln(ic50) refers to the natural log of the ic50.

The term “K_{off}”, as used herein, is intended to refer to the off rate constant, for example, for dissociation of an antibody from the antibody/antigen complex, or for dissociation of an epitope from an MHC haplotype.

The term “K_{d}”, as used herein, is intended to refer to the dissociation constant (the reciprocal of the affinity constant “Ka”), for example, for a particular antibody-antigen interaction or interaction between an epitope and an MHC haplotype.

As used herein, the terms “strong binder” and “strong binding” and “High binder” and “high binding” or “high affinity” refer to a binding pair or describe a binding pair that have an affinity of greater than 2×10⁷M⁻¹ (equivalent to a dissociation constant of 50 nM Kd)

As used herein, the term “moderate binder” and “moderate binding” and “moderate affinity” refer to a binding pair or describe a binding pair that have an affinity of from 2×10⁷M⁻¹ to 2×10⁶M⁻¹.

As used herein, the terms “weak binder” and “weak binding” and “low affinity” refer to a binding pair or describe a binding pair that have an affinity of less than 2×10⁶M⁻¹ (equivalent to a dissociation constant of less than 500 nM Kd) Binding affinity may also be expressed by the standard deviation from the mean binding found in the peptides making up a protein. Hence a binding affinity may be expressed as “−1σ” or <−1σ, where this refers to a binding affinity of 1 or more standard deviations below the mean. A common mathematical transformation used in statistical analysis is a process called standardization wherein the distribution is transformed from its standard units to standard deviation units where the distribution has a mean of zero and a variance (and standard deviation) of 1. Because each protein comprises unique distributions for the different MHC alleles standardization of the affinity data to zero mean and unit variance provides a numerical scale where different alleles and different proteins can be compared. Analysis of a wide range of experimental results suggest that a criterion of standard deviation units can be used to discriminate between potential immunological responses and non-responses. An affinity of 1 standard deviation below the mean was found to be a useful threshold in this regard and thus approximately 15% (16.2% to be exact) of the peptides found in any protein will fall into this category.

The terms “specific binding” or “specifically binding” when used in reference to the interaction of an antibody and a protein or peptide or an epitope and an MHC haplotype means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

As used herein, the term “antigen binding protein” and “antigen binding molecule” refer to proteins or peptides that bind to a specific antigen. “Antigen binding proteins” and ‘Antigen binding molecules” include, but are not limited to, immunoglobulins, including polyclonal, monoclonal, chimeric, single chain, single chain variable fragment (svFC) sequences, immunoglobulin variable regions, humanized antibodies, Fab fragments, F(ab′)2 fragments, and Fab expression libraries.

“Adjuvant” as used herein encompasses various adjuvants that are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, squalene, squalene emulsions, liposomes, imiquimod, keyhole limpet hemocyanins, dinitrophenol, and potentially useful human adjuvants such as BCG (Bacille Calmette-Guerin) and Corynebacterium parvum. In other embodiments a cytokine may be co-administered, including but not limited to interferon gamma or stimulators thereof, interleukin 12, or granulocyte stimulating factor. In other embodiments the peptides or their encoding nucleic acids may be co-administered with a local inflammatory agent, either chemical or physical. Examples include, but are not limited to, heat, infrared light, proinflammatory drugs, including but not limited to imiquimod.

As used herein “immunoglobulin” means the distinct antibody molecule secreted by a clonal line of B cells; hence when the term “100 immunoglobulins” is used it conveys the distinct products of 100 different B-cell clones and their lineages.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “support vector machine” refers to a set of related supervised learning methods used for classification and regression. Given a set of training examples, each marked as belonging to one of two categories, an SVM training algorithm builds a model that predicts whether a new example falls into one category or the other.

As used herein, the term “classifier” when used in relation to statistical processes refers to processes such as neural nets and support vector machines.

As used herein, in the context of mathematical analysis, “neural net”, which is used interchangeably with “neural network” and sometimes abbreviated as NN, refers to various configurations of classifiers used in machine learning, including multilayered perceptrons with one or more hidden layer, support vector machines and dynamic Bayesian networks. These methods share in common the ability to be trained, the quality of their training evaluated, and their ability to make either categorical classifications of non-numeric data or to generate equations for predictions of continuous numbers in a regression mode. Perceptron as used herein is a classifier which maps its input x to an output value which is a function of x, or a graphical representation thereof.

As used herein, the term “principal component analysis”, or as abbreviated “PCA”, refers to a mathematical process which reduces the dimensionality of a set of data (Wold, S., Sjorstrom,M., and Eriksson,L., Chemometrics and Intelligent Laboratory Systems 2001. 58: 109-130.; Multivariate and Megavariate Data Analysis Basic Principles and Applications (Parts I&II) by L. Eriksson, E. Johansson, N. Kettaneh-Wold, and J. Trygg, 2006 2^{nd} Edit. Umetrics Academy). Derivation of principal components is a linear transformation that locates directions of maximum variance in the original input data, and rotates the data along these axes. For n original variables, n principal components are formed as follows: The first principal component is the linear combination of the standardized original variables that has the greatest possible variance. Each subsequent principal component is the linear combination of the standardized original variables that has the greatest possible variance and is uncorrelated with all previously defined components. Further, the principal components are scale-independent in that they can be developed from different types of measurements. The application of PCA generates numerical coefficients (descriptors). The coefficients are effectively proxy variables whose numerical values are seen to be related to underlying physical properties of the molecules. A description of the application of PCA to generate descriptors of amino acids and by combination thereof peptides is provided in PCT US2011/029192 incorporated herein by reference in its entirety. Unlike neural nets PCA do not have any predictive capability. PCA is deductive not inductive.

As used herein, the term “vector” when used in relation to a computer algorithm or the present invention, refers to the mathematical properties of the amino acid sequence.

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors. “Viral vector” as used herein includes but is not limited to adenoviral vectors, adeno-associated viral vectors, lentiviral vectors, retroviral vectors, poliovirus vectors, measles virus vectors, flavivirus vectors, poxvirus vectors, and other viral vectors which may be used to deliver a peptide or nucleic acid sequence to a host cell.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism).

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

A “subject” is an animal such as vertebrate, preferably a mammal such as a human, a bird, or a fish. Mammals are understood to include, but are not limited to, murines, simians, humans, bovines, ovines, cervids, equines, porcines, canines, felines etc.). A “subject affected by cancer” is a cancer patient.

An “effective amount” is an amount sufficient to effect beneficial or desired results. An effective amount can be administered in one or more administrations,

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

As used herein “Complementarity Determining Regions” (CDRs) are those parts of the immunoglobulin variable chains which determine how these molecules bind to their specific antigen. Each immunoglobulin variable region typically comprises three CDRs and these are the most highly variable regions of the molecule. T cell receptors also comprise similar CDRs and the term CDR may be applied to T cell receptors.

As used herein, the term “motif” refers to a characteristic sequence of amino acids forming a distinctive pattern; this may also be expressed as an “amino acid motif”. A “pentamer motif” is a combination of five amino acids, either contiguous to each other or separated by one or more other amino acids The term “Groove Exposed Motif” (GEM) as used herein refers to a subset of amino acids within a peptide that binds to an MHC molecule; the GEM comprises those amino acids which are turned inward towards the groove formed by the MHC molecule and which play a significant role in determining the binding affinity. In the case of human MIC-I the GEM amino acids are typically (1,2,3,9). In the case of MHC-II molecules two formats of GEM are most common comprising amino acids (−3,2,−1,1,4,6,9,+1,+2,+3) and (−3,2,1,2,4,6,9,+1,+2,+3) based on a 15-mer peptide with a central core of 9 amino acids numbered 1-9 and positions outside the core numbered as negative (N terminal) or positive (C terminal).

“Immunoglobulin germline” is used herein to refer to the variable region sequences encoded in the inherited germline genes and which have not yet undergone any somatic hypermutation. Each individual carries and expresses multiple copies of germline genes for the variable regions of heavy and light chains. These undergo somatic hypermutation during affinity maturation. Information on the germline sequences of immunoglobulins is collated and referenced by www.imgt.org [2]. “Germline family” as used herein refers to the 7 main gene groups, catalogued at IMGT, which share similarity in their sequences and which are further subdivided into subfamilies.

“Affinity maturation” is the molecular evolution that occurs during somatic hypermutation during which unique variable region sequences generated that are the best at targeting and neutralizing and antigen become clonally expanded and dominate the responding cell populations.

“Germline motif” as used herein describes the amino acid subsets that are found in germline immunoglobulins. Germline motifs comprise both GEM and TCEM motifs found in the variable regions of immunoglobulins which have not yet undergone somatic hypermutation.

“pMHC” Is used to describe a complex of a peptide bound to an MHC molecule. In many instances a peptide bound to an MHC-I will be a 9-mer or 10-mer however other sizes of 7-11 amino acids may be thus bound. Similarly MHC-II molecules may form pMHC complexes with peptides of 15 amino acids or with peptides of other sizes from 11-23 amino acids. The term pMHC is thus understood to include any short peptide bound to a corresponding MHC.

“Somatic hypermutation” (SHM), as used herein refers to the process by which 5 variability in the immunoglobulin variable region is generated during the proliferation of individual B-cells responding to an immune stimulus. SHM occurs in the complementarity determining regions.

“T-cell exposed motif” (also where abbreviated TCEM), as used herein, refers to the sub set of amino acids in a peptide bound in a MHC molecule which are directed outwards and exposed to a T-cell binding to the pMHC complex. A T-cell binds to a complex molecular space-shape made up of the outer surface MHC of the particular HLA allele and the exposed amino acids of the peptide bound within the MHC. Hence any T-cell recognizes a space shape or receptor which is specific to the combination of HLA and peptide. The amino acids which comprise the TCEM in an MHC-I binding peptide typically comprise positions 4, 5, 6, 7, 8 of a 9-mer. The amino acids which comprise the TCEM in an MHC-II binding peptide typically comprise 2, 3, 5, 7, 8 or −1, 3, 5, 7, 8 based on a 15-mer peptide with a central core of 9 amino acids numbered 1-9 and positions outside the core numbered as negative (N terminal) or positive (C terminal). As indicated under pMHC, the peptide bound to a MHC may be of other lengths and thus the numbering system here is considered a non-exclusive example of the instances of 9-mer and 15 mer peptides.

As used herein “histotope” refers to the outward facing surface of the MHC molecules which surrounds the T cell exposed motif and in combination with the T cell exposed motif serves as the binding surface for the T cell receptor.

As used herein the T cell receptor refers to the molecules exposed on the surface of a T cell which engage the histotope of the MHC and the T cell exposed motif of a peptide bound in said MHC. The natural T cell receptor comprises two protein chains, known as the alpha and beta chain in 95% of human T cells and as the delta and gamma chains in the remaining 5% of human T cells. Each chain comprises a variable region and a constant region. Each variable region comprises three complementarity determining regions or CDRs. In T cells engineered to comprise recombinant T cell receptors the structure of the receptor may be modified, comprising for example fusion molecules or single chains, but the functionality of binding to a specific T cell epitope is retained. “Regulatory T-cell” or “Treg” as used herein, refers to a T-cell which has an immunosuppressive or down-regulatory function. Regulatory T-cells were formerly known as suppressor T-cells. Regulatory T-cells come in many forms but typically are characterized by expression CD4+, CD25, and Foxp3. Tregs are involved in shutting down immune responses after they have successfully eliminated invading organisms, and also in preventing immune responses to self-antigens or autoimmunity.

“uTOPE™ analysis” as used herein refers to the computer assisted processes for predicting binding of peptides to MHC and predicting cathepsin cleavage, described in PCT US2011/029192, PCT US2012/055038, US2014/014523, PCT US2015/039969, PCT US2020/037206, U.S. Pat. Nos. 10,706,955 and 10,755,801, each of which is incorporated herein by reference in its entirety.

“Framework region” as used herein refers to the amino acid sequences within an immunoglobulin variable region which do not undergo somatic hypermutation.

“Isotype” as used herein refers to the related proteins of particular gene family. Immunoglobulin isotype refers to the distinct forms of heavy and light chains in the immunoglobulins. In heavy chains there are five heavy chain isotypes (alpha, delta, gamma, epsilon, and mu, leading to the formation of IgA, IgD, IgG, IgE and IgM respectively) and light chains have two isotypes (kappa and lambda). Isotype when applied to immunoglobulins herein is used interchangeably with immunoglobulin “class”.

“Isoform” as used herein refers to different forms of a protein which differ in a small number of amino acids. The isoform may be a full length protein (i.e., by reference to a reference wild-type protein or isoform) or a modified form of a partial protein, i.e., be shorter in length than a reference wild-type protein or isoform.

“Class switch recombination” (CSR) as used herein refers to the change from one isotype of immunoglobulin to another in an activated B cell, wherein the constant region associated with a specific variable region is changed, typically from IgM to IgG or other isotypes.

“Immunostimulation” as used herein refers to the signaling that leads to activation of an immune response, whether said immune response is characterized by a recruitment of cells or the release of cytokines which lead to suppression of the immune response. Thus, immunostimulation refers to both upregulation or down regulation.

“Up-regulation” as used herein refers to an immunostimulation which leads to cytokine release and cell recruitment tending to eliminate a non self or exogenous epitope. Such responses include recruitment of T cells, including effectors such as cytotoxic T cells, and inflammation. In an adverse reaction upregulation may be directed to a self-epitope.

“Down regulation” as used herein refers to an immunostimulation which leads to cytokine release that tends to dampen or eliminate a cell response. In some instances such elimination may include apoptosis of the responding T cells.

“Frequency class” or “frequency classification” as used herein is used to describe logarithmic based bins or subsets of amino acid motifs or cells. When applied to the counts of TCEM motifs found in a given dataset of peptides a logarithmic (log base 2) frequency categorization scheme was developed to describe the distribution of motifs in a dataset. As the cellular interactions between T-cells and antigen presenting cells displaying the motifs in MHC molecules on their surfaces are the ultimate result of the molecular interactions, using a log base 2 system implies that each adjacent frequency class would double or halve the cellular interactions with that motif Thus, using such a frequency categorization scheme makes it possible to characterize subtle differences in motif usage as well as providing a comprehensible way of visualizing the cellular interaction dynamics with the different motifs. Hence a Frequency Class 2, or FC 2 means 1 in 4, a Frequency class 10 or FC 10 means 1 in 2¹⁰ or 1 in 1024. In other embodiments the frequency classification of the TCEM motif in the reference dataset is described by the quantile score of the TCEM in the reference dataset. Quantile scores are used, but is not limited to, applications where the reference dataset is the human proteome or a microbial proteome. “Frequency class” or “frequency classification” may also be applied to cellular clonotypic frequency where it refers to subgroups or bins defined by logarithmic based groupings, whether log base 2 or another selected log base.

A “rare TCEM” as used herein is one which is completely missing in the human proteome or present in up to only five instances in the human proteome.

“Adverse immune response” as used herein may refer to (a) the induction of immunosuppression when the appropriate response is an active immune response to eliminate a pathogen or tumor or (b) the induction of an upregulated active immune response to a self-antigen or (c) an excessive up-regulation unbalanced by any suppression, as may occur for instance in an allergic response.

“Clonotype” as used herein refers to the cell lineage arising from one unique cell. In the particular case of a B cell clonotype it refers to a clonal population of B cells that produces a unique sequence of IGV. The number of B cells that express that sequence varies from singletons to thousands in the repertoire of an individual. In the case of a T cell it refers to a cell lineage which expresses a particular TCR. A clonotype of cancer cells all arise from one cell and carry a particular mutation or mutations or the derivates thereof. The above are examples of clonotypes of cells and should not be considered limiting.

As used herein “epitope mimic” or “TCEM mimic” is used to describe a peptide which has an identical or overlapping TCEM, but may have a different GEM. Such a mimic occurring in one protein may induce an immune response directed towards another protein which carries the same TCEM motif. This may give rise to autoimmunity or inappropriate responses to the second protein.

“Cytokine” as used herein refers to a protein which is active in cell signaling and may include, among other examples, chemokines, interferons, interleukins, lymphokines, granulocyte colony-stimulating factor, tumor necrosis factor and programmed death proteins.

As used herein “oncoprotein” means a protein encoded by an oncogene which can cause the transformation of a cell into a tumor cell if introduced into it. Examples of oncoproteins include but are not limited to the early proteins of papillomaviruses, polyomaviruses, adenoviruses and herpesviruses, however oncoproteins are not necessarily of viral origin. “MHC subunit chain” as used herein refers to the alpha and beta subunits of MHC molecules. A MHC II molecule is made up of an alpha chain which is constant among each of the DR, DP, and DQ variants and a beta chain which varies by allele. The MHC I molecule is made up of a constant beta macroglobulin and a variable MHC A, B or C chain.

As used here in “virome” comprises the viruses present in a human subject, latently chronically or during acute infection, or a sub set thereof made up of viruses of a particular taxonomic group or of the viruses located in a particular tissue or organ.

“Immunoglobulinome” as used herein refers to the total complement of immunoglobulins produced and carried by any one subject.

As used herein the term “repertoire’ is used to describe a collection of molecules or cells making up a functional unit or whole. Thus, as one non limiting example, the entirely of the B cells or T cells in a subject comprise its repertoire of B cells or T cells. The entirety of all immunoglobulins expressed by said B cells are its immunoglobulinome or the repertoire of immunoglobulins. A collection of proteins or cell clonotypes which make up a tissue sample, an individual subject or a microorganism may be referred to as a repertoire.

As used herein “mutated amino acid” refers to the appearance of an amino acid in a protein that is the result of a nucleotide change, a missense mutation, or an insertion or deletion or fusion.

“Splice variant” as used herein refers to different proteins that are expressed from one gene as the result of inclusion or exclusion of particular exons of a gene in the final, processed messenger RNA produced from that gene or that is the result of cutting and re-annealing of RNA or DNA.

“TRAV” as used herein refers to the T cell receptor alpha variable region family or allele subgroups and “TRBV” refers to T cell receptor beta variable region family or allele subgroups as described in IMGT http://imgt.org/IMGTrepertoire/Proteins/index.php #C http://imgt.org/IMGTrepertoire/Proteins/taballeles/human/TRA/TRAV/Hu_TRAVall.html TRAV comprises at least 41 subgroups, with some having sub-subgroups. TRBV comprises at least 30 subgroups. Most combinations of alpha and beta variable region subgroups are encountered. “hTRAV” refers to human TRAV.

As used here in a “receptor bearing cell” is any cell which carries a ligand binding recognition motif on its surface. In some particular instances a receptor bearing cell is a B cell and its surface receptor comprises an immunoglobulin variable region, said immunoglobulin variable region comprising both heavy and light chains which make up said receptor. In other particular instances a receptor bearing cell may be a T cell which bears a receptor made up of both alpha and beta chains or both delta and gamma chains. Other examples of a receptor bearing cell include cells which carry other ligands such as, in one particular non limiting example, a programmed death protein of which there are multiple isoforms.

As used herein the term “bin” refers to a quantitative grouping and a “logarithmic bin” is used to describe a grouping according to the logarithm of the quantity.

As used herein “immunotherapy intervention” is used to describe any deliberate modification of the immune system including but not limited to through the administration of therapeutic drugs or biopharmaceuticals, radiation, T cell therapy, application of engineered T cells, which may include T cells linked to cytotoxic, chemotherapeutic or radiosensitive moieties, checkpoint inhibitor administration, cytokine or recombinant cytokine or cytokine enhancer, including but not limited to a IL-15 agonist, cathepsin inhibitor, microbiome manipulation, vaccination, B or T cell depletion or ablation, or surgical intervention to remove any immune related tissues.

As used herein “immunomodulatory intervention” refers to any medical or nutritional treatment or prophylaxis administered with the intent of changing the immune response or the balance of immune responsive cells. Such an intervention may be delivered parenterally or orally or via inhalation. Such intervention may include, but is not limited to, a vaccine including both prophylactic and therapeutic vaccines, a biopharmaceutical, which may be from the group comprising an immunoglobulin or part thereof, a T cell stimulator, checkpoint inhibitor, or suppressor, an adjuvant, a cytokine, a cytotoxin, receptor binder, an enhancer of NK (natural killer) cells, an interleukin including but not limited to variants of IL 15, superagonists, cathepsin inhibitor, and a nutritional or dietary supplement. The intervention may also include radiation or chemotherapy to ablate a target group of cells. The impact on the immune response may be to stimulate or to down regulate.

“Checkpoint inhibitor” or “checkpoint blockade” as used herein refers to a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include, but are not limited to, PD-1/PD-L1 and CTLA-4/B7-1/B7-2.

As used herein the “cluster of differentiation” proteins refers to cell surface molecules providing targets for immunophenotyping of cells. The cluster of differentiation is also known as cluster of designation or classification determinant and may be abbreviated as CD. Examples of CD proteins include those listed at www.uniprot.org/docs/cdlist As used herein “tumor associated mutations” refers to all nucleotide or amino acid mutations detected in a tumor. In some cases the tumor associated mutations are commonly found within many patients with a particular tumor type. In other cases tumor associated mutations may be unique to a specific patient. In other instances different patients may carry different tumor associated mutations are in the same protein.

“Pattern” as used herein means a characteristic or consistent distribution of data points.

As used herein a “frequency pattern” is a data set that displays the frequency of TCEMs in a repertoire of proteins from a proteome associated with an individual subject as compared to the frequency of those TCEMs in a reference database. Particular TCEMs, or groups of TCEMs, within the subject's repertoire may occur at the same, lower or higher frequencies than the corresponding TCEMs in the reference database. The frequency pattern allows identification and categorization of unique TCEMs and/or patterns of TCEMs (i.e., unique features of unique TCEM features). The term “frequency pattern” as used herein is also used to describe the distribution of cellular clonotypes within a repertoire of cells from an individual subject, as compared to the frequency of the cellular clonotypes in a reference database. Particular clonotypes, or groups of clonotypes, within the subject's repertoire may occur at the same, lower or higher frequencies than the corresponding cellular clonotypes in the reference database. The frequency pattern allows identification and categorization of unique patterns of clonotypes. In some embodiments, a “frequency class” or “frequency classification” is assigned to a TCEM motif or to a cellular clonotype based on its frequency as described elsewhere herein.

As used herein “clonotypic diversity” refers to the distribution of the total number of cells in a repertoire among all unique clonotypes in a repertoire. Hence, if a repertoire has 1 million cells, but these comprise 400,000 of clonotype 1 and 600,000 of clonotype 2, the repertoire has a low clonotypic diversity. If the 1 million cells are distributed as 10 each of 100,000 unique clonotypes the repertoire has a high clonotypic diversity.

As used herein “many to one” describes a relationship in which one protein or peptide sequence is encoded be many different synonymous nucleotide sequences.

As used herein “presentome” refers to the peptides bound in MHC and presented on the surface of antigen presented cells. Mass spectroscopy detects some but not all peptides which are part of the presentome.

“Neoantigen” as used herein refers to a novel epitope motif or antigen created as the result of introduction of a mutation into an amino acid sequence. Thus, a neoantigen differentiates a wildtype protein from its mutant-bearing tumor protein homolog, when such mutant is presented to T cells or B cells.

“Tumor specific antigen” or “tumor specific epitope” is used herein to designate an epitope or antigen that differentiates a mutated tumor protein from its unmutated wildtype homologue. Thus, a neoantigen is one type of tumor specific antigen.

As used herein “driver” mutations are those which arise very early in tumorogeneis and are causally associated with the early steps of cell dysregulation. Driver mutations are shared by all clonal offspring arising from the initial tumor cells and offer some additional fitness benefit to the clonal line within its microenvironment. In contrast passenger mutations are those somatic mutations which arise during the differentiation of the tumor and which offer no particular benefit of fitness to the cell. Passengers may serve as biomarkers on tumor cells and may enable some immune evasion. Passenger mutations may differ at different time points in its development and among different parts of a tumor or among metastases. “Driver and passenger” are terms largely interchangeable with “trunk and branch” mutations.

“Bespoke peptides” or “bespoke vaccine” as used herein refers to a peptide or neoantigen or a combination of peptides, or nucleic acid encoding peptides, that are tailored or personalized specifically for an individual patient, taking into account that patient's HLA alleles. A bespoke peptide or bespoke vaccine is also referred to herein as a “personalized peptide”, “personalized peptide vaccine”, “personalized neoepitope vaccine” or “personalized vaccine”.

As used herein “TCGA” refers to The Cancer Genome Atlas (www.cancer.gov/about-nci/organization/ccg/research/structural-genomics/tcga) As used herein a “polyhydrophobic amino acid” refers to a short chain of natural amino acids which are hydrophobic. Examples include, but are not limited to, leucines, isoleucines or tryptophans where these are assembled in multimers of 5-15 repeats of any one such amino acid. As a non-limiting example, a poly leucine comprising 8 leucines would be an example of a polyhydrophobic amino acid.

A “lipid core peptide system”, as used herein, refers to subunit vaccine comprising a lipoamino acid (LAA) moiety which allows the stimulation of immune activity A combination of T cell stimulating epitopes or T and B cell stimulating epitopes are linked to a LAA. Multiple different constructs can be created with of different spatial orientaiion or LAA lengths (e.g. C12 2-amino-D,L-dodecanoic acid or C16, 2-amino-D,L-hexadecanoic acid,). When dissolved in a standard phosphate buffer LCP particles form and the particles facilitate uptake by antigen presenting cells. Different LAA chain lengths lead to different particle sizes.

As used herein, a “BAM” file is a compressed binary version of a Sequence Alignment File “SAM” file wherein the all nucleotides are aligned to a reference genome. A “BAM slice” is a subset of the entire genome defined by genome coordinates. The HLA locus is located on Chromosome 6. In one particular instance a BAM slice is defined to contain just the HLA locus.

“Antigen presenting cell” as used herein refers to cells which are capable of presentation of peptides to T cells bound to MHC molecules. This includes but is not limited to the so called “professional” antigen presenting cells comprising but not limited to dendritic cells, B cells, and macrophages, but also the so called non-professional antigen presenting cells which carry MHC molecules.

“Parenteral” as used herein refers to any direct injection into the body, including but not limited to intradermal, subcutaneous, intramuscular, intraperitoneal and intravenous injection.

“Non-parenteral” as used herein refers to delivery per os to any point in the gastrointestinal tract, to the mucosa of the upper and lower respiratory tract, rectal mucosa or genitourinary tract. Topical application to the skin is also non-parenteral

“Originating peptide” as used herein refers to a naturally occurring peptide, whether mutated or not, which comprises a T cell exposed motif and an amino acid of interest therein, that is used as the basis for designing a peptide with desired binding affinity for a particular MHC allele.

“Proposed peptide” as used herein refers to the peptide with desired binding affinity for a particular MHC allele which is designed by changing the amino acids not in the T cell exposed motif and then selected from a list of such peptides for potential inclusion in a vaccination regimen.

As used herein, the term “motif” refers to a characteristic sequence of amino acids forming a distinctive pattern, this may also be expressed as an “amino acid motif”. A “pentamer motif is a combination of five amino acids, either contiguous to each other or separated by one or more other amino acids

“HUGO” as used herein refers to the Human Genome Organisation Gene Nomenclature Committee at the European Bioinformatics Institute (world wide web at genenames.org) which assigns a name and an approved gene symbol to each gene. Examples of HUGO gene names included herein are BCAN (brevican), TBXT (brachyury), NCAN (neurocan), VCAN (versican), ACAN (aggrecan), FN1 (fibronectin)

“Tumor associated antigen” as used herein refers to an antigen found in a protein that is not mutated or changed from a normal sequence in a tumor but which may be expressed on the surface of a tumor cell and may be expressed at higher levels in a tumor.

“Glioma” as used herein refers to any tumor of glial cell origin, including but not limited to gliomas, glioblastoma, glioblastoma multiforme, astrocytoma, ependymoma, and oligodendroglioma. Gliomas may occur intracranially or in the brainstem. Glioma also refers to gliomas of specific cranial nerves such as an optic nerve glioma. Gliomas may be high grade or low grade, and may be described by a World Health Organization Grade I-IV according to the WHO Classification of Tumors of the Central Nervous System (https://tumourclassification.iarc.who.int).

“Chordoma” as used herein refers to a tumor derived from a remnant of the notochord and characterized by continued expression and/or increased copy number of the brachyury gene.

“Adoptive cell transfer” or “ACT” as used herein refers to the introduction of allogenic or autologous cells into a subject. Adoptive cell transfer of T cells may include the selection of epitope-specific T cells clones and expansion of these for re-administration to a subject. ACT may also include “chimeric antigen receptor bearing cells” otherwise known as “CAR-T” or the transfer of “engineered T cells” or “TCR-T” which have been modified to carry recombinant T cell receptors cognate for the T cell epitopes in a protein of interest or displayed on a cell of interest.

“Perineuronal net” as used herein, and not to be confused with “neural net” above, refers to extracellular matrix structures, typically comprising chondroitin sulphate proteoglycans that surround axons and dendrites and their nodes.

“Cognate” when used here in in relation to a T cell refers to a T cell bearing a receptor that binds to a specific T cell exposed motif in the context of an HLA histotope.

As used herein, the term “mRNA transcript expression” metric refers to the total number of sequencing reads for a particular gene that has been corrected for the length in kilobases of the mRNA transcript of the particular gene and the number of total reads for the particular sequenced sample. This metric is generally referred to as FPKM (fragments per kilobase per million reads) or RPKM (reads per kilobase per million reads).

As used herein the term “normalized mRNA transcript expression” refers to fitting the log 10 transformed FPKM with a SHASH continuous distribution function and then transforming the fit into standard deviation units of a zero mean and unit variance normal distribution. After this transformation the metric conforms to the “68-95-99.7 rule”, also known as the “empirical rule” that describes the percentage of values that lie within an interval estimate in a normal distribution: 68%, 95%, and 99.7% of the values lie within one, two, and three standard deviations of the mean, respectively.

### DESCRIPTION OF THE INVENTION

Tumors arise from genetic mutations in certain genes which lead to loss or gain of function and which have evaded immune surveillance. Very often a tumor microenvironment is not conducive to the development of a cytotoxic immune response that can halt progression or eliminate a tumor. Two approaches have been taken towards the development of immunization strategies which can stimulate an active T cell response to tumors: the use of tumor specific neoantigens and the targeting of tumor associated antigens that are expressed by tumors but are not mutated. The challenge is to identify T cell epitopes, and hence vaccination targets, that elicit a cytotoxic response directed to the tumor cells but without collateral damage to adjacent normal cells and with minimal autoimmune responses to other tissues. With neoantigens this separation is achieved by targeting the T cell epitopes comprising the mutations, whether missense, indel, splice variant or fusion which are unique to the tumor. Tumor associated antigens have been employed in the past, including epitopes in PMEL, MAGE1, TRP1, ERBB2, WT1 and others. However, expression of these antigens is not restricted to particular tissues, is present in normal tissues, and in brain tumors and particularly glioblastoma, these genes are rarely upregulated.

The present invention relates to tumor associated antigens that are upregulated in brain tumors. Brevican is expressed by glial cells and is upregulated in gliomas. It therefore provides a promising target for immunotherapy of glioma patients. The present invention provides a means of selection of peptides for targeting CD8+ and CD4+ to brevican by application of natural and of heteroclitic peptides personalized to a particular subject. This allows the selection and design of peptides suitable for stimulation of T cell responses in subjects of any MHC I or MHC II HLA. In addition to the stimulation of T cells cognate for brevican in vivo, such T cells may be harvested and their numbers expanded for re-administration to further enhance the response in the subject. The T cell receptors from the T cells showing a specific response may also be sequenced and engineered into T cells of allogenic or autologous sources.

In another embodiment the present invention identifies linear B cell epitopes in brevican which, when administered with adjacent T cell helper epitopes, or when delivered with an appropriate adjuvant, may elicit an antibody response. Such epitopes may also be the basis for development of antibodies that serve as diagnostic reagents. Immunoglobulin variable regions, or antigen binding molecules that are components thereof, targeting these B cell epitopes can serve as a component of CAR-T cells to target T cells to a particular tumor cell displaying brevican.

The overall goal of the inventions provided herein are to down-regulate brevican in order to slow or arrest tumor progression, and to alter the tumor immune microenvironment.

### Brevican

The extracellular matrix of the brain is an important component of the tumor microenvironment and differs significantly from other tissues. A unique component of the brain extracellular matrix is brevican [3], a chondroitin sulfate proteoglycan, structurally similar to versican, neurocan and aggrecan. It is a 911 amino acid secreted protein encoded in Chromosome 1. Brevican is also known as Brain Enriched Hyaluronan Binding protein or BEHAB (Hugo symbol BCAN). Brevican is identified in Uniprot as PGCB_HUMAN Q96GW7 and we refer to the isoform 1 as SEQ ID NO: 1:

sp|Q96GW7|PGCB_HUMAN Brevican core protein
OS = Homo sapiens OX = 9606 GN = BCAN PE = 1
SV = 2
MAQLFLPLLAALVLAQAPAALADVLEGDSSEDRAFRVRIAGDAPLQGVL
GGALTIPCHVHYLRPPPSRRAVLGSPRVKWTFLSRGREAEVLVARGVRV
KVNEAYRFRVALPAYPASLTDVSLALSELRPNDSGIYRCEVQHGIDDSS
DAVEVKVKGVVFLYREGSARYAFSFSGAQEACARIGAHIATPEQLYAAY
LGGYEQCDAGWLSDQTVRYPIQTPREACYGDMDGFPGVRNYGVVDPDDL
YDVYCYAEDLNGELFLGDPPEKLTLEEARAYCQERGAEIATTGQLYAAW
DGGLDHCSPGWLADGSVRYPIVTPSQRCGGGLPGVKTLFLFPNQTGFPN
KHSRENVYCFRDSAQPSAIPEASNPASNPASDGLEAIVTVTETLEELQL
PQEATESESRGAIYSIPIMEDGGGGSSTPEDPAEAPRTLLEFETQSMVP
PTGFSEEEGKALEEEEKYEDEEEKEEEEEEEEVEDEALWAWPSELSSPG
PEASLPTEPAAQEESLSQAPARAVLQPGASPLPDGESEASRPPRVHGPP
TETLPTPRERNLASPSPSTLVEAREVGEATGGPELSGVPRGESEETGSS
EGAPSLLPATRAPEGTRELEAPSEDNSGRTAPAGTSVQAQPVLPTDSAS
RGGVAVVPASGDCVPSPCHNGGTCLEEEEGVRCLCLPGYGGDLCDVGLR
FCNPGWDAFQGACYKHESTRRSWEEAETQCRMYGAHLASISTPEEQDFI
NNRYREYQWIGLNDRTIEGDFLWSDGVPLLYENWNPGQPDSYFLSGENC
VVMVWHDQGQWSDVPCNYHLSYTCKMGLVSCGPPPELPLAQVFGRPRLR
YEVDTVLRYRCREGLAQRNLPLIRCQENGRWEAPQISCVPRRPARALHP
EEDPEGRQGRLLGRWKALLIPPSSPMPGP

Brevican forms a major constituent of the brain extracellular matrix, but is not expressed elsewhere [4, 5]. It is abundant in the neonatal brain and during early development, but expressed at lower levels in the normal adult brain [6]. Brevican is expressed only by glial cells [7]. The brevican molecule comprises a N terminal hyaluronan binding domain, and a C terminal domain that contains an EGF type repeat, a lectin domain and a complement regulatory like domains. There is also a shorter, 671 amino acid, non-secreted GPI anchored form, which has an anchor sequence starting from amino acid position 647 of the full-length isoform [3]. There are also multiple isoforms of brevican based on differential splicing, cleavage and glycosylation, and glycosylation patterns may be differ in gliomas [8, 9]. Cleavage of the full-length protein by metalloproteinase ADAMTS4 prior to Ser401 releases an N terminal fragment which enhances EGFR signaling and also binds to fibronectin, facilitating invasion of tumor cells. [9-12]. In the normal brain brevican forms part of the perineuronal net coating large axons, especially in the hippocampus [13]. Absence of brevican does not impede brain development and mice deficient in brevican show no evidence of loss of learning and memory [14, 15].

Brevican is upregulated in humans during embryonic and immediate post-natal gliogenesis and highly expressed until about 8 years of age; then downregulated to consistently low levels in the adult brain. Brevican is strongly expressed in immature oligodendrocyte precursor cells in the promyelinating phase but absent once these cells mature, although it is expressed in mature astrocytes [16]. In the mature brain brevican forms part of the extracellular nodal matrix of large diameter axons, contributing to regulation of conductance [17]. Other molecules such as versican, BRAL1, and tenascin fulfill the same role for smaller diameter axons and provide this function in brevican deficient mice. While the brevican N-terminal domain may bind hyaluronan, the C-terminal domain can bind to either tenascin-R or sulfated cell surface glycolipids [17].

Brevican may be upregulated in the adult brain in response to trauma [6, 7, 18]. Increases in brevican have been reported in Alzheimer's disease indicating that brevican may be an immunotherapy target here also [5, 13]. Similarly, brevican may be upregulated, along with changes in other extracellular matrix proteins in schizophrenia [19]. The increased expression arises from increased transcription rather than any change in copy number.

Brevican is highly upregulated in gliomas, i.e. all tumors of glial origin, including astrocytoma, oligodendroglioma, and glioblastoma, where its level of expression is correlated with invasion and progression. Interestingly, it is not expressed in brain metastases of other tumor types or tumors of non-glial origin [4]. Expression in gliomas averages about 700% that detected in normal adult brain. Brevican expression is detected by immunohistochemistry in histological sections of multiple grades of glioma and glioblastoma, but not in benign brain tumors or in other tumor types [20]. Within tumors, brevican is most highly expressed in regions showing other tumorigenic markers such as OLIG2 and CD133 [10]. An isoform lacking most glycosylation is most upregulated in advanced grade gliomas [8, 20]. This isoform is bound to tumor cell membranes [9]. A highly sialylated version is also expressed [8]. The role of brevican in invasiveness of gliomas is attributed to its binding to fibronectin and collagen type IV as well as hyaluronic acid binding [11]. Brevican only promotes invasion once cleaved by ADAMTS4 and only the N terminal cleavage product is essential to invasion [11, 12, 21].

In vitro experiments with multiple glioma cell lines have shown that growth and invasiveness is correlated with brevican overexpression. Experimental knockdown down of brevican regulates proliferation and invasion and spread of brevican-expressing cells in vitro and in xenograft transplants in mice [10, 20]. Conversely, adding brevican by transfection of non-invasive cell lines increases their invasiveness [20]. This points to a key role of brevican in invasiveness and growth of tumors and that targeting brevican may have beneficial effects in slowing progression.

As alterations in the extracellular matrix are key to tumor cell migration and invasiveness, targeting proteins which bring about changes in the extracellular matrix provides a means of limiting invasion [22]. As brevican is uniquely expressed in glioma/glioblastoma and has a pro-invasive role that appears to assist later progression it offers a specific tumor associated target for immunotherapy of gliomas. Gliomas, and particularly glioblastomas, comprise a large and particularly serious group of cancers that have proven largely recalcitrant to small molecule drugs, radiation and other forms of intervention. Thus, alternative approaches to intervention, and particularly interventions which may be applied early after diagnosis of a glioma or glioblastoma are urgently needed.

In the present invention we identify epitopes in brevican suitable for targeting by peptide vaccination and provide methods to optimize T cell stimulation in individual subjects based on their HLA alleles. As those skilled in the art will appreciate, a peptide epitope may be presented to T cells either by delivery of a peptide or by administration of a nucleic acid that encodes that peptide.

From an immunological perspective the primary isoform of the brevican molecule comprises three regions, in addition to the signal peptide. Most T cell epitopes are located in the N terminal region (N terminal cleavage product) from aa 24 to 350 and in the C terminal between 650 and the C terminal at 911. The most dense B cell epitope region is between 350 and 650, although less dominant B cell epitopes lie outside this range. Given that the molecule is cleaved by ADAMTS4 prior to position 401, and the role the molecule plays in tumor invasiveness depends on the N terminal cleavage product, T cell epitopes for targeting are selected from the region 24-401. B cell epitopes are selected also from the cleaved N terminal sequence, but also from the C terminal region between 401 and 650, where they may direct antibodies and antigen binding molecules to membrane bound epitopes. Brevican comprises two purported hyaluronan binding sites at 167-168 and 265-266.

### T Cell Epitopes

The goal of T cell immunotherapy has been primarily to activate CD8+ cytotoxic T cells which will target tumor cells, but also to stimulate CD4+ T helper cells to enhance CD8+ responses and the establishment of a T cell memory. The combination of both CD8+ and CD4+ peptides is therefore desirable. A preferred combination of MHC I and MHC II binding peptides to ensure stimulation and formation of a memory are peptides that are closely adjacent or overlapping. Stimulation of CD4+ T helper cells may also enhance B cell responses. In selecting T cell epitopes to target in brevican, we take into consideration the presence of peptides that will bind MHC I and MHC II, both naturally or sub-dominantly and select peptides based on the HLA alleles of each subject affected by a tumor. Priming of T cells is critical to establishment of functional CD8+ cytotoxic cells. In a desired embodiment the peptide-MHC binding affinity is in the range of 20-200 nanomolar. In other embodiments a binding affinity of from 200 nM to 1000 nM may be sufficient to induce a T cell response. The former falls within the range of 1-2 standard deviation units of all binding of peptides in the protein; the latter is in the range of 1-0.5 standard deviation units of all binding of peptides in the protein. A higher binding affinity (the top 2.5 standard deviation units and above, equivalent to the 2.5% highest affinity peptides in the protein for any particular HLA allele) is more likely cause persistent stimulation and lead to T cell exhaustion and anergy [23-26].

Stimulation of T cell responses to selected peptide epitopes, or the nucleic acids that encode them, may be achieved in various ways. In one desired embodiment the peptides are delivered directly to the subject by vaccination. Here the selected peptides, or their encoding nucleic acids, may be delivered parenterally, by injection intradermally, subcutaneously, intramuscularly or intravenously. In some preferred embodiments delivery is by microneedle or microneedle patch. In yet other embodiments the peptides may be delivered non-parenterally by means of an oral tablet, capsule or other formulation. In some preferred embodiments, the peptides are particulate. In others they are encased in a lipid drug delivery system. In preferred embodiments the lipid drug delivery system comprises a solid lipid nanoparticle, an emulsion or microemulsion, a self-emulsifying drug delivery system [27], a nanocapsule or a liposome.

In an alternative mode of delivery T cell stimulating peptides may be contacted in vitro with dendritic cells derived from the patient, and following culture of the dendritic cells, these antigen presenting cells are readministered to the subject to stimulate a T cell response. In an alternative embodiment, following vaccination either directly or ex vivo to dendritic cells, T cells drawn from the subject are identified that are responsive to the selected peptides in vitro and these cells are harvested and expanded in vitro to re-administer to the subject or, alternatively, to enable engineering of their T cell receptors into other T cells. A vaccination regimen for T cell stimulation to the epitopes of interest may be achieved by a primary vaccination of one, two three or more administrations, followed by periodic boosters.

In some embodiments, the peptide or nucleic acid vaccination may be accompanied by other immunotherapy or immunomodulatory interventions. In one embodiment, vaccination may be accompanied by, or followed by, administration of a checkpoint inhibitor which serves to upregulate T cells broadly, including those stimulated by the selected peptides.

In yet other embodiments, the identification of T cells responding to specific epitopes identified by the invention provides the opportunity to harvest these T cells and expand them in vitro, and also to derive the sequences of their T cell receptors for engineering as recombinant TCR into additional T cells.

Prior to selecting T cell epitope peptides for administration to a particular subject, the T cell exposed motifs that the proposed peptides comprise are evaluated to identify any T cell exposed motif that may also occur in peptides in other proteins of the human proteome and which, in that context, would also have a high binding affinity to the particular subject's alleles. These are reviewed for the potential of adverse off-target responses and removed from the selection. This process is described in more detail in PCT APPL. US2021/062140 incorporated herein by reference in its entirety.

Immunization to stimulate a T cell response to brevican is an approach which lends itself to early treatment of gliomas, and particularly glioblastomas. Given the almost uniform upregulation of brevican in glioblastomas, provided that HLA alleles of a subject are determined, peptides can be selected or designed very rapidly and administered shortly after clinical diagnosis, without awaiting the longer process of surgery and biopsy sequencing, or where surgery and/or biopsy may be contraindicated. Once biopsy sequencing is available this initial intervention may be supplemented by neoepitope vaccination and adoptive cell transfer.

### B Cell Epitopes

The B cell epitopes within brevican provide options for other approaches to targeting glioma cells. As described below in Example 5, linear B cell epitopes are detected at the N terminal extreme of the secreted brevican and also in the region between amino acid positions 350 and 401 immediately preceding the cleavage site. In addition, dominant B cell epitopes are found in the C terminal side of the cleavage site between positions 401 and 650. While the epitopes on the N terminal side may result in antigen binding molecules targeting the cleavage product necessary for invasiveness, those on the C terminal side of the cleavage site may be retained on the glial cell membrane and are present in the anchored isoform of brevican. Peptides comprising B cell epitopes may be administered along with T helper epitopes, either as an extended peptide spanning both epitopes, or separately, or with alternate T helper peptides or proteins to elicit brevican specific antibodies. Antibodies elicited by the vaccination may coat the brevican bearing cells directly and induce an antibody dependent cytotoxicity. Another mode of utilization of antibodies to the B cell epitopes is to engineer the immunoglobulin variable regions, or components thereof, into CAR-T cells. As brevican is widely upregulated in gliomas and glioblastomas, this provides a mode of attack applicable broadly to many brain tumor cases. B cell epitopes, with adjacent or alternate T helper epitopes or adjuvants, may be used to generate antibody reagents by immunization of non-human animals. These subjects can also serve as a source of antigen binding molecules, such as scFV, for use in engineering CAR-T cells.

### Adoptive Cell Transfer

Most approaches to immunotherapy depend on the generation of effective tumor specific T cells. While these may be stimulated directly by immunization with neoantigen or tumor associated T cell epitopes, effector T cells may also be transferred to the affected subject by adoptive cell transfer. This may comprise the harvesting of tumor-antigen specific T cells from the subject, either naturally occurring or stimulated by immunization, followed by their expansion in culture. Alternatively, the transferred T cells may comprise engineered T cell receptors designed to specifically target epitopes on the tumor. The first of these approaches to be developed was CAR-T cells (chimeric antigen T cells) [28]. This approach targets B cell epitopes displayed on tumors. Here, a component of an immunoglobulin, comprising at least the scFV component of the variable region is attached by a linker to a T cell “armed” with various costimulatory and signaling domains. The source of the T cells may be either allogenic or autologous to the patient. CAR-T cells have proven their efficacy in hematologic cancers, where antibodies directed to common antigens such as CD19 facilitate a common design and hematologic cells can be replenished by stem cell transfer and natural regeneration. Selecting appropriate tumor specific targets for solid tumors have proven far more challenging, given the relative lack of tumor specific targets shared among tumors and the unique nature of mutations in each subject's tumor. Thus, tumor specific antibody epitopes that have shared characteristics across multiple subjects and tumors are a highly desirable as away to target CAR-T cells.

Efforts to target CAR-T to gliomas has involved various targeted, including EGFRviii, HER2 and IL13Ra2 [29, 30] Another chondroitin sulfate proteoglycan CPSG4, which has essentially no homology with brevican, has been identified as a potential target in glioblastoma, and has had some success in melanoma. Its use in glioblastoma is limited by the potential of off-target effects in other tissues. Expression of CPSG4 is correlated with invasiveness in melanoma, breast cancer, head and neck squamous cell carcinomas and mesothelioma by mechanisms similar to that of brevican in the unique setting of the brain. [31].

A second approach for anti-tumor adoptive cell transfer is to engineer naïve T cells with recombinant T cell receptors (TCR). This allows T cells to be redirected to T cell epitopes displayed on tumors. TCR sequences are determined in T cells harvested from tumors and shown to be tumor specific. Then gene transfer of the specific TCR into T cells, which may be autologous or allogenic in origin. The transfer may be vector mediated, utilizing for instance adenoviral, retroviral, lentiviral or adeno associated viral vectors, or may be accomplished by non-vector means such as transposons or mRNA. TCR-T cell engineering has largely focused on tumor associated antigens such as MART1, WT1, NY-ESO1 (CTAG1A) and MAGE [32]. Alternative targets, and especially those common to multiple subjects are needed. In the present invention we provide brevican epitopes for the stimulation of T cell clones specific to brevican which may then be used as the source for TCR sequences for engineering into naïve T cells, expansion thereof and subsequent transfer.

### Selection of Peptides for Formulation and Administration

In some embodiments, therefore, the present invention provides methods for formulation for parenteral delivery of epitopes of interest in brevican by several routes, including but not limited to intradermally, intramuscularly and intratumorally. In yet other embodiments the invention provides methods to deliver such peptide vaccines non-parenterally, including but not limited to orally. The criteria for selection of peptides to optimize formulation have been addressed in PCT APPL. US2021/062140 (incorporated herein by reference in its entirety). Briefly, the main considerations are to select peptides with consideration of their stability and solubility [33]. Chemical changes such as oxidation and deamidation comprise one source of instability problems. Peptides comprising methionine, tryptophan, histidine, cystine and tyrosine are most prone to oxidation, whereas those comprising asparagine and glutamine are prone to deamidation. In one embodiment therefore, to reduce oxidation, peptides are selected in which amino acids from the group comprising methionine, tryptophan, histidine, cystine and tyrosine are excluded in the groove exposed motif. In yet another embodiment, to reduce deamidation, peptides are selected in which asparagine and glutamine are not present in the groove exposed motif. Exclusion of cysteine has the additional benefit in reducing cross linking between peptides by formation of disulfide bonds.

Physical challenges to stability include the formation of aggregates or micelles, adsorption to surfaces and denaturation due to extremes of temperature or pH. Various strategies have been developed to mitigate each of these including, but not limited to, the use of surfactants and lower concentrations, adjusting salt concentrations and pH (to reduce aggregations), polymer excipients such as polysorbate 80, selection of appropriate containers (to mitigate adsorption), addition of salts or metal ions and control of pH (to reduce denaturation), addition of buffers, selection of storage temperature (to reduce hydrolysis) and addition of antioxidants and chelating agents (to reduce oxidation).

Biological challenges to stability include enzymatic degradation and intestinal permeability. Strategies have been developed to mitigate both the above including but not limited to the use of enteric drug delivery systems and permeation enhancers. To overcome the enzymatic and pH-dependent degradation of peptides in the stomach, in addition to permeability issues and the potential for degradation via first pass metabolism, formulation strategies, such as enzymatic activity inhibitors, permeation enhancers, enteric coatings, and carrier molecules, can be employed

Solubility of peptides is at a minimum at the isoelectric point. Hence optimization of pH, salt concentration and ionic strength are non-limiting examples of approaches to improve solubility. An assessment of peptide solubility in aqueous solvents can be made by determining the polarity and the partition coefficient. A determination of the octanol:water partition coefficient is another useful guide as it could predict the molecules solubility and permeability. In some embodiments therefore peptides are selected based on their index of polarity. In other embodiments the selection is based on the partition coefficient or the log thereof (log P), and in preferred embodiments the partition coefficient of octanol:water.

In some particular embodiments peptides are selected to include highly polar amino acids in their groove exposed motif positions. In some particularly preferred embodiments peptides are selected to include amino acids selected from the group comprising arginine, lysine, glutamic acid or aspartic acid in the groove exposed motifs.

A number of stabilizing excipients may be included to assist in stabilizing during formulation including, but not limited to polysorbate 20, polysorbate 80 and sodium dodecyl sulfate, pluronic 107, polyethylene glycol, dextran, hydroxyethyl starch, ascorbic acid, salts of sulfurous acid, and thiols, ethylene glycol, glycerol, glucose, mannitol. Therefore, in some embodiments peptides are formulated with one or more stabilizing excipients.

Molecular weight is another consideration in manufacturability of peptides. Peptides selected from the proteins of interest described herein may be up to about 25-30 amino acids. Peptides which are personalized by designing the groove exposed motifs to optimize HLA binding are typically up to 15 or 16 amino acids for MHC II binding peptides and 8-10 amino acids for MHC I binding peptides.

Peptides selected by the methods described herein therefore have a low molecular weight. In some embodiments the selected vaccinal peptides are under or equal to 4000 Da. In preferred embodiments the molecular weight of each selected vaccinal peptide is less than or equal to 2000 Da; in a highly preferred embodiment the peptide molecular weight is less than or equal to 1500 Da.

While peptides are one desirable mode of delivery, in alternative embodiments the selected peptides may be encoded in nucleic acids for expression in vivo.

### Selection of Adjuvants

Peptides in isolation are not readily taken up by antigen presenting cells without the addition of an adjuvant. In some cases, the adjuvant effect is a function of the form in which peptides are administered, including, but not limited to, when peptides are delivered as an emulsion, particulate, liposome, virosome, or glucan particle. In other instances a peptide vaccine may be administered with an adjuvant selected from the following non-limiting examples: lipid A analogues (e.g. poly I:C), imidazoquinolines 9e.g. imiquimod), CpG, saponins, C type lectin ligands, CD1d ligands 9 e.g. a-galactosylceramide), aluminum salts (e.g. aluminum hydroxide), emulsions (e.g. MF59), and many variants thereof [34, 35]. Adjuvants may act in many ways, by enhancing antigen uptake by antigen presenting cells, by activating toll receptors, activating inflammasomes, enhancing immune cell recruitment and by increasing presentation of antigen to T cells [36]. A further adjuvant used with neoantigen peptides has been granulocyte stimulating factor [35]. Combinations of adjuvants may be used together. In the case of peptide vaccines, enhancing antigen uptake by antigen presenting cells, both professional and non-professional, is the most critical function of an adjuvant.

### EXAMPLES

### Example 1: Upregulation of Brevican in Glioma

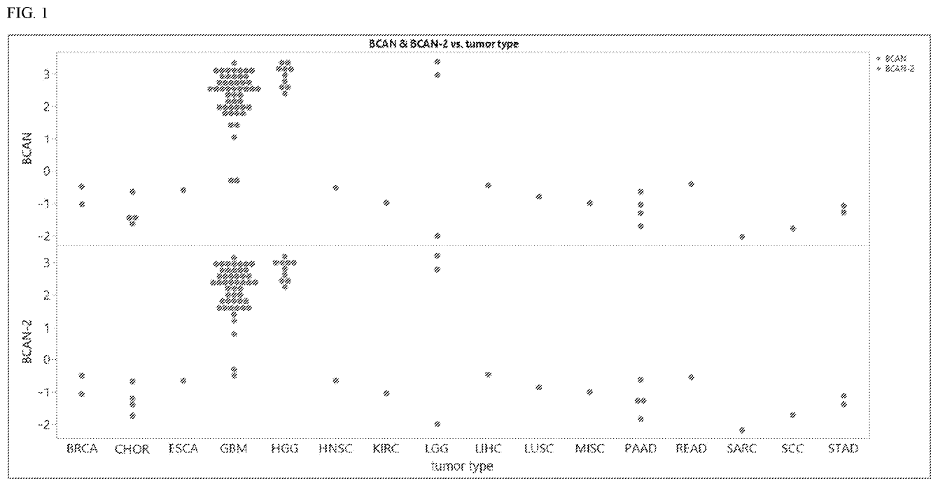

As noted in the background section above, it has been widely reported that brevican is upregulated in gliomas [3]. Our own observations confirm this to be the case. FIG. 1 shows in expression of brevican in a series of 49 glioblastoma, 10 high grade and 3 low grade gliomas compared to other tumors of both intra and extracranial origin.

As is known to those skilled in the art, bulk mRNA transcript enumeration is carried out using a bioinformatic process that has been designed to tally transcription of different genes. The resulting data is enumerated as the FPKM (fragments per kilobase per million total reads) that normalizes the metric for both the length of the transcribed coding region and the number of total reads in the bulk sample detected by the sequencing machine. The relative mRNA expression is then expressed as the normalized mRNA expression as show in FIG. 1, which shows BCAN expression in most glioblastoma cases as at least 1, and in some cases at least 2 standard deviations above the mean of the tumor proteome, or in the top 15% or 2.5% of expressed transcripts.

### Example 2: T Cell Epitopes in Brevican

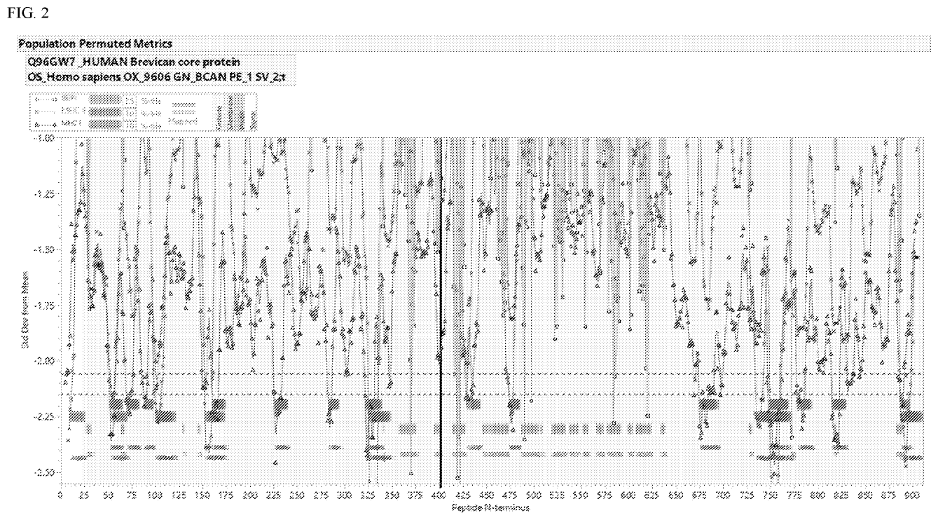

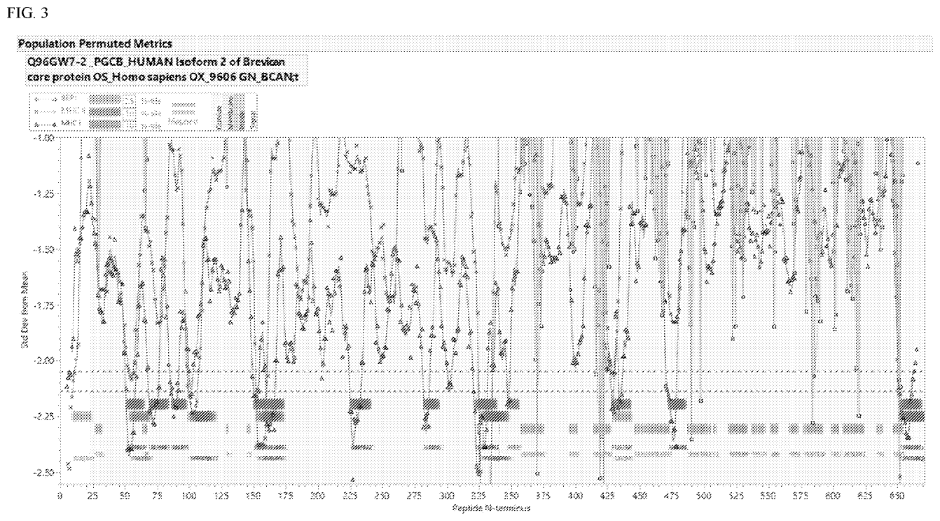

FIGS. 2 and 3 identify those regions of the N terminal cleaved component with MHC I and MHC II binding in the top 10% for a permuted average of human HLA alleles in the 911 amino acid isoform 1 of brevican and the 671 amino acid anchored isoform. Six extended peptides were identified as encompassing the highest binding regions. Two extended peptides were selected to span the hyaluronan binding sites. These are shown in Table 1 with the predicted binding affinity for two common MHC I alleles as examples.

TABLE 1

| SEQ ID NO: | Dominant T cell epitope regions |
|---|---|
| 2 | GALTIPCHVHYLRPPPSRRA |
| 3 | NEAYRFRVALPAYPASLTDVSLALS |
| 4 | KGVVFLYREGSARYAFSF |
| 5 | ARYAFSFSGAQEACARIGAH |
| 6 | DLNGELFLGDPPEKLTLEEARAYCQERGAE |
| 7 | GLPGVKTLFLFPNQTGFPNKHSRF |

Within these peptides T cell exposed motifs of particular interest and the peptides spanning them were identified. These are shown in Tables 2 and 3 together with binding affinity for A0801, A1101, A0201, A2402 and DRB1 0101, DRB1 0301and DRB1_0401. Predicted binding affinity was computed for the peptides spanning each T cell exposed motifs for 31 MHC I A alleles, 31 MHC I B alleles, 8 MHC I C alleles, and 24 DRIB alleles, but selected examples for a few alleles are shown here in the interests of space, and thus are not considered limiting.

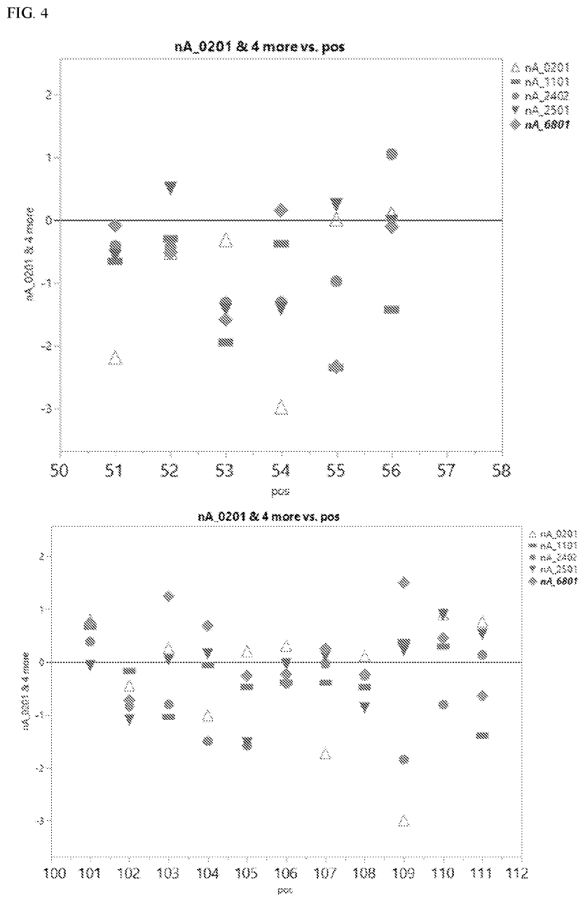

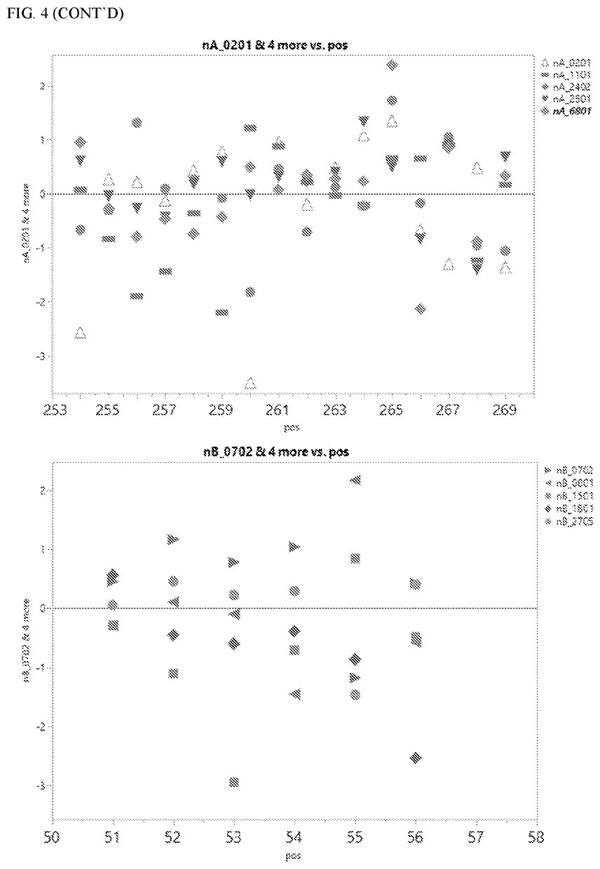

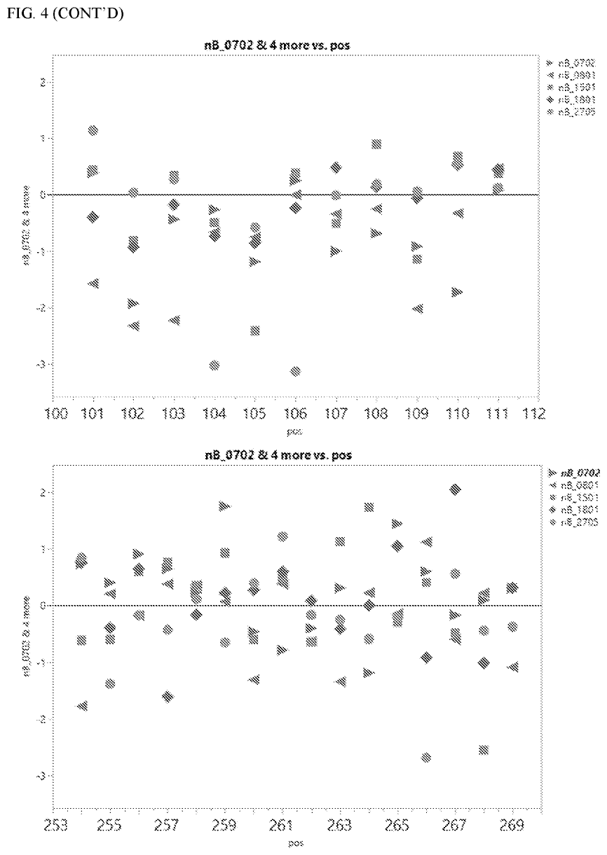

Table 2 and 3 show binding in standard deviation units (SD) below the mean for the isoform 1 of brevican (Uniprot Q96GW7). The desired binding affinity to achieve immunostimulation is between −1 and −2.25 SD units, or within the top 16% but not as high affinity as the top 0.61%. Thus Table 2 shows that certain peptides are suitable for the alleles shown. For instance, SEQ ID 8, SEQ ID 19, SEQ ID 35 and SEQ ID 36 are desirable for A0201 and SEQ ID 10, SEQ ID 11, SEQ ID 16, SEQ ID 17, SEQ ID 20, and SEQ ID 36 are desirable for A2402. Conversely, SEQ ID 11, SEQ ID 20, SEQ ID 31and SEQ ID 40 are extremely high binders more likely to produce T cell exhaustion with allele A0201. Similarly, in Table 3 for MHC II, SEQ IDs 82-85 are desirable for DRB1_0101 and DRB1_0401. However, SEQ ID 109 has an excessive affinity for DRB 1_0101 and SEQ ID 110 is an excessive high affinity for DRB1_0401. For allele-peptide combinations where affinity is predicted between 0 and −1 SD units, a heteroclitic “personal” peptide may be designed as described below by placement of alternative amino acids in the positions flanking the T cell exposed motifs. FIG. 4 also shows differential binding various alleles.

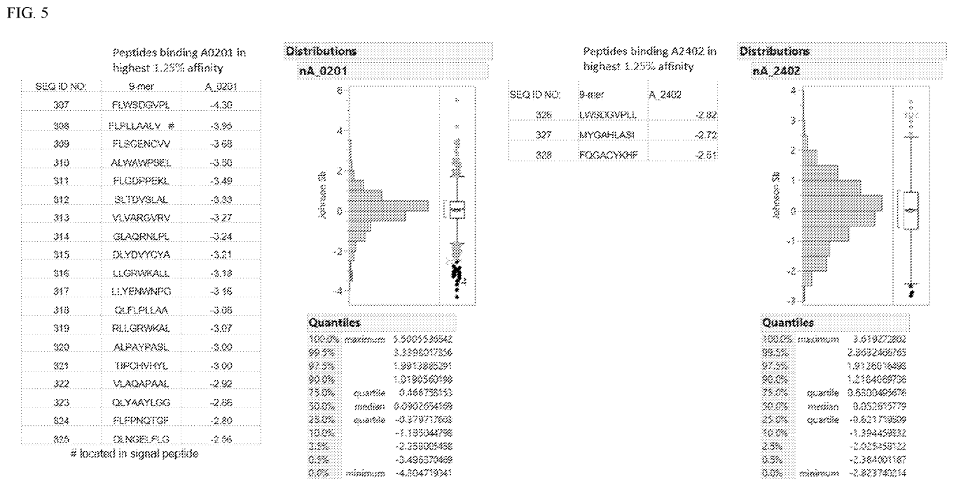

A ranking of predicted highly bound binding of peptides in brevican for common alleles is shown in FIG. 5 and Table 4, indicating peptides that are in the top approximately 0.61% of binding affinity of those in the protein for two example alleles and considered likely to induce exhaustion. It should be noted that while these peptides may be less desirable for the two alleles shown (A0201 and A2402), they may have an affinity for other alleles that falls within the desirable range. Such highly bound peptides may be more readily detected by mass spectroscopy than lower binding peptides. Accordingly, in some embodiments, the methods and the compositions of the present invention exclude the following peptides: FLGDPPEKL (SEQ ID NO:311); ALWAWPSEL (SEQ ID NO:329); and FLWSDGVPL (SEQ ID NO:330).

The Tables 2 and 3 also reflect the omission of certain T cell exposed motifs and peptides due to potential adverse cross reactivity with other critical human proteins and high binding affinity across many alleles. For instance ˜˜˜PAYPA˜(SEQ ID 306) is found in peptides with high binding affinity for NMDE4 in multiple MHC I alleles.

TABLE 2
Preferred brevican MHC I binding peptides
are those with binding of −1 to −2.5 SD units

| | | | TCEM | | | | |
|---|---|---|---|---|---|---|---|
| SEQ | | | SEQ | | | | |
| ID NO: | 9-mer | TCEM I | ID NO: | A_0201 | A_2402 | A_1101 | A_0801 |
| 8 | GALTIPCHV | ~~~TIPCH~ | 42 | −2.21 | −0.42 | −0.67 | −0.40 |
| 9 | ALTIPCHVH | ~~~IPCHV~ | 43 | −0.53 | −0.44 | −0.30 | 0.11 |
| 10 | LTIPCHVHY | ~~~PCHVH~ | 44 | −0.33 | −1.32 | −1.96 | −0.59 |
| 11 | TIPCHVHYL | ~~~CHVHY~ | 45 | −3.00 | −1.31 | −0.39 | −1.28 |
| 12 | IPCHVHYLR | ~~~HVHYL~ | 46 | 0.00 | −0.99 | −2.36 | 0.22 |
| 13 | PCHVHYLRP | ~~~VHYLR~ | 47 | 0.09 | 1.04 | −1.44 | −0.53 |
| 14 | EAYRFRVAL | ~~~RFRVA~ | 48 | −0.45 | −0.84 | −0.17 | −2.45 |
| 15 | AYRFRVALP | ~~~FRVAL~ | 49 | 0.25 | −0.80 | −1.04 | −0.74 |
| 16 | YRFRVALPA | ~~~RVALP~ | 50 | −1.02 | −1.50 | −0.05 | −1.27 |
| 17 | RFRVALPAY | ~~~VALPA~ | 51 | 0.20 | −1.58 | −0.47 | 0.13 |
| 18 | FRVALPAYP | ~~~ALPAY~ | 52 | 0.30 | −0.41 | −0.39 | 0.22 |
| 19 | RVALPAYPA | ~~~LPAYP~ | 53 | −1.73 | −0.03 | −0.39 | −0.90 |
| 20 | ALPAYPASL | ~~~AYPAS~ | 54 | −3.00 | −1.84 | 0.38 | −1.32 |
| 21 | GVVFLYREG | ~~~FLYRE~ | 55 | −0.26 | 1.45 | −0.24 | −0.68 |
| 22 | VVFLYREGS | ~~~LYREG~ | 56 | 0.36 | 0.47 | −0.46 | −1.28 |
| 23 | VFLYREGSA | ~~~YREGS~ | 57 | −0.31 | −0.29 | 0.81 | −2.59 |
| 24 | ARYAFSFSG | ~~~AFSFS~ | 58 | −0.45 | −0.59 | 0.22 | −0.76 |
| 25 | RYAFSFSGA | ~~~FSFSG~ | 59 | 0.34 | −1.08 | 1.01 | −0.71 |
| 26 | FSFSGAQEA | ~~~SGAQE~ | 60 | −1.34 | 1.17 | 0.47 | −0.78 |
| 27 | SFSGAQEAC | ~~~GAQEA~ | 61 | 0.35 | −1.47 | 0.21 | −0.08 |
| 28 | DLNGELFLG | ~~~GELFL~ | 62 | −2.56 | −0.67 | 0.07 | −0.95 |
| 29 | ELFLGDPPE | ~~~LGDPP~ | 63 | 0.42 | 0.26 | −0.36 | 0.24 |
| 30 | LFLGDPPEK | ~~~GDPPE~ | 64 | 0.77 | −0.08 | −2.20 | −0.08 |
| 31 | FLGDPPEKL | ~~~DPPEK~ | 65 | −3.49 | −1.82 | 1.22 | −2.01 |
| 32 | LGDPPEKLT | ~~~PPEKL~ | 66 | 0.94 | 0.46 | 0.87 | 0.55 |
| 33 | KLTLEEARA | ~~~LEEAR~ | 67 | −1.30 | 1.05 | 0.90 | −1.21 |
| 34 | LTLEEARAY | ~~~EEARA~ | 68 | 0.48 | −0.96 | −1.23 | 0.20 |
| 35 | TLEEARAYC | ~~~EARAY~ | 69 | −1.37 | −1.06 | 0.16 | −0.51 |
| 36 | GLPGVKTLF | ~~~GVKTL~ | 70 | −1.79 | −2.22 | −0.41 | 0.50 |
| 37 | LPGVKTLFL | ~~~VKTLF~ | 71 | −0.04 | −1.87 | 0.61 | −1.08 |
| 38 | PGVKTLFLF | ~~~KTLFL~ | 72 | −1.08 | −2.24 | −1.21 | −0.61 |
| 39 | LFLFPNQTG | ~~~FPNQT~ | 73 | 0.13 | −0.33 | 0.55 | −0.98 |
| 40 | FLFPNQTGF | ~~~PNQTG~ | 74 | −2.80 | −2.29 | 0.04 | −2.21 |
| 41 | LFPNQTGFP | ~~~NQTGF~ | 75 | 0.07 | −1.06 | −0.62 | −0.04 |

Predicted binding affinity is shown in standard deviation units about the mean of all peptides in the protein.

TABLE 3
Preferred brevican MHC II binding peptides
are those with binding of −1 to −2.5 SD units

| peptide | | TCEM II | | | | |
|---|---|---|---|---|---|---|
| SEQ | | SEQ | TCEM | | | |
| ID NO: | peptide | ID NO: | IIa | DRB1_0101 | DRB1_0401 | DRB1_0301 |
| 76 | GALTIPCHVHYLRPP | 112 | IP~H~HY | −0.51 | −0.56 | −1.59 |
| 77 | ALTIPCHVHYLRPPP | 113 | PC~V~YL | −0.66 | −1.29 | −2.40 |
| 78 | LTIPCHVHYLRPPPS | 114 | CH~H~LR | −0.07 | −1.34 | −2.28 |
| 79 | TIPCHVHYLRPPPSR | 115 | HV~Y~RP | −0.25 | −0.83 | −1.39 |
| 80 | IPCHVHYLRPPPSRR | 116 | VH~L~PP | −0.78 | −1.61 | −1.72 |
| 81 | PCHVHYLRPPPSRRA | 117 | HY~R~PP | −1.04 | −0.77 | −0.99 |
| 82 | NEAYRFRVALPAYPA | 118 | RF~V~LP | −2.22 | −2.24 | −0.54 |
| 83 | EAYRFRVALPAYPAS | 119 | FR~A~PA | −1.74 | −2.23 | −0.39 |
| 84 | AYRFRVALPAYPASL | 120 | RV~L-AY | −1.87 | −2.50 | −0.05 |
| 85 | YRFRVALPAYPASLT | 121 | VA~P~YP | −2.29 | −1.70 | 0.47 |
| 86 | RFRVALPAYPASLTD | 122 | AL~A~PA | −1.82 | −1.47 | −0.16 |
| 87 | FRVALPAYPASLTDV | 123 | LP~Y~AS | −2.52 | −1.67 | 0.25 |
| 88 | RVALPAYPASLTDVS | 124 | PA~P~SL | −1.52 | −1.06 | 1.34 |
| 89 | VALPAYPASLTDVSL | 125 | AY~A~LT | −1.21 | −0.67 | 0.62 |
| 90 | ALPAYPASLTDVSLA | 126 | YP~S~TD | −1.75 | −1.56 | 1.17 |
| 91 | LPAYPASLTDVSLAL | 127 | PA~L~DV | −2.11 | −1.53 | 0.21 |
| 92 | KGVVFLYREGSARYA | 128 | FL~R~GS | −1.37 | −2.24 | −1.84 |
| 93 | GVVFLYREGSARYAF | 129 | LY~E~SA | −1.47 | −1.74 | −1.10 |
| 94 | VVFLYREGSARYAFS | 130 | YR~G~AR | −1.66 | −2.06 | −1.89 |
| 95 | VFLYREGSARYAFSF | 131 | RE~S~RY | −1.24 | −0.41 | −0.87 |
| 96 | ARYAFSFSGAQEACA | 132 | FS~S~AQ | −1.26 | −1.11 | 0.69 |
| 97 | DLNGELFLGDPPEKL | 133 | EL~L~DP | −0.49 | −0.35 | −0.03 |
| 98 | LNGELFLGDPPEKLT | 134 | LF~G~PP | −1.14 | −1.56 | −0.73 |
| 99 | NGELFLGDPPEKLTL | 135 | FL~D~PE | −1.50 | −1.59 | −1.61 |
| 100 | GELFLGDPPEKLTLE | 136 | LG~P~EK | −1.79 | −0.06 | −1.21 |
| 101 | ELFLGDPPEKLTLEE | 137 | GD~P~KL | −0.93 | 0.00 | −1.09 |
| 102 | PEKLTLEEARAYCQE | 138 | TL~E~RA | 0.05 | 0.72 | −1.37 |
| 103 | GLPGVKTLFLFPNQT | 139 | VK~L~LF | −0.65 | −0.19 | 0.57 |
| 104 | LPGVKTLFLFPNQTG | 140 | KT~F~FP | −0.97 | −1.32 | 0.64 |
| 105 | PGVKTLFLFPNQTGF | 141 | TL~L~PN | −0.59 | −1.49 | 0.56 |
| 106 | GVKTLFLFPNQTGFP | 142 | LF~F~NQ | −1.83 | −1.79 | 0.49 |
| 107 | VKTLFLFPNQTGFPN | 143 | FL~P~QT | −2.56 | −1.93 | −1.50 |
| 108 | KTLFLFPNQTGFPNK | 144 | LF~N~TG | −2.59 | −2.43 | −1.92 |
| 109 | TLFLFPNQTGFPNKH | 145 | FP~Q~GF | −3.19 | −1.99 | −3.45 |
| 110 | LFLFPNQTGFPNKHS | 146 | PN~T~FP | −2.11 | −2.77 | −1.13 |
| 111 | FLFPNQTGFPNKHSR | 147 | NQ~G~PN | −0.48 | −1.15 | −0.68 |

TABLE 4
Brevican peptides with predicted binding in top
approximately 1.25% affinity for A0201 and A2402

| SEQ | | | SEQ | | |
|---|---|---|---|---|---|
| ID NO: | 9-mer | A_0201 | ID NO: | 9-mer | A_2402 |
| 307 | FLWSDGVPL | −4.30 | 326 | LWSDGVPLL | −2.82 |
| 308 | FLPLLAALV # | −3.95 | 327 | MYGAHLASI | −2.72 |
| 309 | FLSGENCVV | −3.68 | 328 | FQGACYKHF | −2.51 |
| 310 | ALWAWPSEL | −3.50 | | | |
| 311 | FLGDPPEKL | −3.49 | | | |
| 312 | SLTDVSLAL | −3.33 | | | |
| 313 | VLVARGVRV | −3.27 | | | |
| 314 | GLAQRNLPL | −3.24 | | | |
| 315 | DLYDVYCYA | −3.21 | | | |
| 316 | LLGRWKALL | −3.18 | | | |
| 317 | LLYENWNPG | −3.16 | | | |
| 318 | QLFLPLLAA | −3.08 | | | |
| 319 | RLLGRWKAL | −3.07 | | | |
| 320 | ALPAYPASL | −3.00 | | | |
| 321 | TIPCHVHYL | −3.00 | | | |
| 322 | VLAQAPAAL | −2.92 | | | |
| 323 | QLYAAYLGG | −2.86 | | | |
| 324 | FLFPNQTGF | −2.80 | | | |
| 325 | DLNGELFLG | −2.56 | | | |

# located in signal peptide.
Binding affinity is shown in standard deviations about the mean of all peptides in brevican.

### Example 3: Design of Peptides with Desired Binding Affinity

The development of tumor vaccines that comprise peptides with a selected desired affinity for the subject's alleles builds on methods previously described to precisely predict MHC binding, identify and analyze T cell exposed motifs and generate peptides with altered binding affinity (See PCT Appl. U.S. Ser. No. 14/41,523, PCT Appl. U.S. Ser. No. 15/39,969, and PCT Appl U.S. Ser. No. 17/21,781, all of which are incorporated herein by reference in their entirety).

### Identification of Relevant Pep/Ide Positions.

In some instances, a protein of interest may comprise peptides at strategic locations, with functional significance such as a receptor or substrate binding site, where such peptides also have a predicted binding affinity for a particular subject's MHC alleles. In this situation the naturally occurring peptide may be selected to induce an immune response. Considerations may include selecting a peptide in a location which will be presented in a mature protein and which has functional significance, as well as selection of adjacent or overlapping peptides which provide both CD8+ T cell stimulation and CD4+T helper functions to achieve full maturation and memory cell development of the CD8+ cytotoxic T cells. In some situations, it is desirable to avoid a peptide which has an excessive binding affinity and is prone to lead to exhaustion or anergy of T cells.

However, in other situations the predicted binding for the particular HLA alleles carried by the patient of a peptide found in strategic location in the protein maybe inadequate to ensure stimulation. In other instances, enhancing binding may be needed to break tolerance. In these situations, one or more customized peptides may be designed to optimize binding for the particular subject and favor the stimulation of a cognate clonal population of T cells.

A T cell epitope peptide comprises an exposed amino acid motif that engages the T cell receptor in the context of the MHC histotope and a flanking motif which determines MHC binding. For MHC I the T cell exposed (TCEM) positions are 4,5,6,7 or 8 of a 9-mer peptide and for MHC II at positions 2,3,5,7,8 of the 9-mer core of a 15 mer. This identifies TCEM IIA; TCEM IIB positions are at −1,3,5,7,8.

To optimize binding for a particular HLA allele we first calculate the predicted binding affinity of all sequential peptide positions in the protein of interest and then selected those peptides with relevant TCEM. For each of the selected peptides comprising a TCEM of interest, a bank of peptides is generated by randomly varying the flanking amino acids, and recalculating the new binding affinity for each allele of interest. For a 9-mer with a pentamer exposed TCEM, this implies up to 160,000 (204) different peptides could be generated, each with a different binding affinity. For practical purposes a bank of 10,000 peptides is usually sufficient to provide peptides within the range of binding affinity desired. For MHC II we opt to vary only those amino acids outside the core 9 mer peptide comprising the TCEM, as the intercalated amino acids which are in pocket (groove exposed) positions affect binding but may also influence the positioning of the exposed amino acids. A further practical consideration is manufacturability of the peptide. A score is generated based on the polarity of the constituent amino acids and only peptides likely to be soluble were put forward as candidates. Sufficient peptides can be generated to prevent this from becoming a limitation.

### Example 4: Example of Selection of Personalized Peptides in Brevican for Use in Vaccine

Each brain tumor patient has a unique combination of MHC I and MHC II HLA alleles. For some HLA alleles a naturally brevican peptide will provide an appropriate MHC binding affinity to achieve T cell stimulation, as shown in Example 2. But, as shown in Tables 2 and 3, for others, the binding may be insufficient to cause T cell stimulation, or the binding may be so high as to be more likely to induce T cell exhaustion and contribute to an anergic tumor microenvironment. In these situations, where some level of sub-dominant binding to the subject's MHCs exists in the natural peptide. T cell clones cognate for the T cell exposed motifs of choice can be stimulated by a modified heteroclitic peptide that provides a more desired level of MHC binding. In some subjects this approach may be necessary for some alleles while a natural peptide provides stimulation for other alleles. This example demonstrates the design of a set of T cell stimulating peptides from brevican for a particular subject with HLAs comprising A2501, A6801, B1801, B4402 and DRB1_0701 DRB1_0801. It will be appreciated that this is one example of such a process for one subject, and it not considered limiting, as a subject of a different HLA genotype could have been similarly addressed. Tables 4 and 5 shows the originating peptide and how personalized peptides have been designed to provide a more desired binding affinity. FIG. 4 shows examples of differential binding by different alleles and indicates why design of a peptide with increased binding may be desirable for a particular patient.

In the following Table 5 and 6 the predicted binding affinity is shown in standard deviation units about the mean for all peptides in the brevican protein. The tables show the predicted binding affinity for the natural peptide comprising the same T cell exposed motif and hence how the placement of alternate amino acids in the flanking regions increase the binding affinity.

TABLE 5
Personalized brevican peptides MHC I

| | | | | | | | | | | Originating peptide | | | | | |
|---|---|---|---|---|---|---|---|---|---|---|---|---|---|---|---|
| Novel peptide | | | | | | | | | | | origi- | | | | |
| Pro- | Novel | | | | | | | | | | nating | | | | |
| posed | peptide | | | | Binding affinity | | | | | origi- | peptide | Binding affinity | | | |
| pep- | SEQ ID | TCEM | TCEM | | in SD units | | | | po- | nating | SEQ | in SD units | | | |
| tide | NO: | core | SEQ ID | Allele | A2501 | A6801 | B1801 | B4402 | larity | peptide | ID NO: | A2501 | A6801 | B1801 | B4402 |
| GERTI | 148 | ~~~TIPCH~ | SEQ ID | B_4402 | | | | −2.07 | −1.05 | GALTI | 8 | | | | −0.23 |
| PCHT | | | 42 | | | | | | | PCHV | | | | | |
| DLTIP | 149 | ~~~IPCHV~ | SEQ ID | A_6801 | | −2.06 | | | 0.42 | ALTIP | 9 | | −0.53 | | |
| CHVR | | | 43 | | | | | | | CHVH | | | | | |
| DVRPC | 150 | ~~~PCHVH~ | SEQ ID | A_2501 | −2.02 | | | | 0.16 | LTIPC | 10 | −1.43 | | | |
| HVHF | | | 44 | | | | | | | HVHY | | | | | |
| GAPPC | 151 | ~~~PCHVH~ | SEQ ID | A_6801 | | −2.05 | | | −0.51 | LTIPC | 10 | | −1.60 | | |
| HVHR | | | 44 | | | | | | | HVHY | | | | | |
| LERPC | 152 | ~~~PCHVH~ | SEQ ID | B_1801 | | | −2.02 | | −1.04 | LTIPC | 10 | | | −0.60 | |
| HVHE | | | 44 | | | | | | | HVHY | | | | | |
| VSYCH | 153 | ~~~CHVHY~ | SEQ ID | A_2501 | −2.01 | | | | 0.31 | TIPCH | 11 | −1.41 | | | |
| VHYR | | | 45 | | | | | | | VHYL | | | | | |
| VDPHV | 154 | ~~~HVHYL~ | SEQ ID | B_1801 | | | −2.07 | | 0.14 | IPCHV | 12 | | | −0.87 | |
| HYLR | | | 46 | | | | | | | HYLR | | | | | |
| GEEHV | 155 | ~~~HVHYL~ | SEQ ID | B_4402 | | | | 1.98 | −0.29 | IPCHV | 12 | | | | −1.14 |
| HYLA | | | 46 | | | | | | | HYLR | | | | | |
| EVFRF | 156 | ~~~RFRVA~ | SEQ ID | A_2501 | −2.07 | | | | 0.71 | EAYRF | 14 | −1.09 | | | |
| RVAP | | | 48 | | | | | | | RVAL | | | | | |
| GAFRF | 157 | ~~~RFRVA~ | SEQ ID | A_6801 | | −1.99 | | | 0.10 | EAYRF | 14 | | −0.72 | | |
| RVAR | | | 48 | | | | | | | RVAL | | | | | |
| EDFRF | 158 | ~~~RFRVA~ | SEQ ID | B_1801 | | | −2.03 | | −1.13 | EAYRF | 14 | | | −0.93 | |
| RVAE | | | 48 | | | | | | | RVAL | | | | | |
| TAGVA | 159 | ~~~VALPA~ | SEQ ID | A_2501 | −2.01 | | | | 0.56 | RFRVA | 17 | −1.53 | | | |
| LPAR | | | 51 | | | | | | | LPAY | | | | | |
| DGLVA | 160 | ~~~VALPA~ | SEQ ID | A_6801 | | −2.06 | | | 0.70 | RFRVA | 17 | | −0.26 | | |
| LPAR | | | 51 | | | | | | | LPAY | | | | | |
| PDFVA | 161 | ~~~VALPA~ | SEQ ID | B_1801 | | | −2.05 | | 0.85 | RFRVA | 17 | | | −0.85 | |
| LPAR | | | 51 | | | | | | | LPAY | | | | | |
| AEGVA | 162 | ~~~VALPA~ | SEQ ID | B_4402 | | | | −2.00 | 0.86 | RFRVA | 17 | | | | −0.17 |
| LPAA | | | 51 | | | | | | | LPAY | | | | | |
| DEALP | 163 | ~~~LPAYP~ | SEQ ID | B_4402 | | | | −2.01 | −0.05 | RVALP | 19 | | | | −0.44 |
| AYPA | | | 53 | | | | | | | AYPA | | | | | |
| REAFL | 164 | ~~~FLYRE~ | SEQ ID | B_4402 | | | | −2.03 | −0.77 | GVVFL | 21 | | | | −0.94 |
| YREP | | | 55 | | | | | | | YREG | | | | | |
| IAILY | 165 | ~~~LYREG~ | SEQ ID | A_6801 | | −2.03 | | | 0.46 | VVFLY | 22 | | −0.62 | | |
| REGR | | | 56 | | | | | | | REGS | | | | | |
| GGTYR | 166 | ~~~YREGS~ | SEQ ID | A_2501 | −2.07 | | | | −0.95 | VFLYR | 23 | −0.41 | | | |
| EGSF | | | 57 | | | | | | | EGSA | | | | | |
| LEFYR | 167 | ~~~YREGS~ | SEQ ID | B_1801 | | | −2.04 | | 0.00 | VFLYR | 23 | | | −0.20 | |
| EGSV | | | 57 | | | | | | | EGSA | | | | | |
| DALSG | 168 | ~~~SGAQE~ | SEQ ID | A_2501 | −2.08 | | | | −1.93 | FSFSG | 26 | −0.37 | | | |
| AQER | | | 60 | | | | | | | AQEA | | | | | |
| REVGA | 169 | ~~~GAQEA~ | SEQ ID | B_1801 | | | −2.00 | | −1.96 | SFSGA | 27 | | | −0.31 | |
| QEAT | | | 61 | | | | | | | QEAC | | | | | |
| EFALG | 172 | ~~~LGDPP~ | SEQ ID | A_6801 | | −2.03 | | | −0.48 | ELFLG | 29 | | −0.75 | | |
| DPPR | | | 63 | | | | | | | DPPE | | | | | |
| AEGPP | 173 | ~~~PPEKL~ | SEQ ID | B_4402 | | | | −2.05 | −1.92 | LGDPP | 32 | | | | −0.20 |
| EKLE | | | 66 | | | | | | | EKLT | | | | | |

TABLE 6
Personalized brevican peptides MHC II

| Novel peptides | | | | | | | | | Originating peptides | | | | |
|---|---|---|---|---|---|---|---|---|---|---|---|---|---|
| | | | | | MHC II | | | | | | MHC II Binding | | |
| | Novel | | | | Binding Novel | | | | | | originating | | |
| | peptide | | | | peptides | | | | origi- | peptide | peptides | | |
| Proposed | SEQ ID | TCEM | TCEM II | | DRB1 | DRB1 | DRB3 | po- | nating | SEQ ID | DRB1 | DRB1 | DRB3 |
| peptide | NO: | core | SEQ ID | Allele | 0701 | 0801 | 0101 | larity | peptide | NO: | 0701 | 0801 | 0101 |
| YVREPCHV | 174 | PC~V~YL | SEQ ID | DRB3 | | | −2.02 | 0.95 | ALTIPCHV | 77 | | | −1.00 |
| HYLALPA | | | 113 | 0101 | | | | | HYLRPPP | | | | |
| RFIVHVHY | 175 | HV~Y~RP | SEQ ID | DRB1 | | −2.06 | | 0.63 | TIPCHVHY | 79 | | −1.05 | |
| LRPDPPA | | | 115 | 0801 | | | | | LRPPPSR | | | | |
| GKRNVHYL | 176 | VH~L~PP | SEQ ID | DRB3 | | | −2.00 | −0.28 | IPCHVHYL | 80 | | | −0.82 |
| RPPYAVA | | | 116 | 0101 | | | | | RPPPSRR | | | | |
| AKVLHYLR | 177 | HY~R~PP | SEQ ID | DRB1 | | −2.04 | | 0.98 | PCHVHYLR | 81 | | −1.52 | |
| PPPPPLY | | | 117 | 0801 | | | | | PPPSRRA | | | | |
| GDYRRFRV | 178 | RF~V~LP | SEQ ID | DRB1 | −2.02 | | | 0.16 | NEAYRFRV | 82 | −1.64 | | |
| ALPAAPV | | | 118 | 0701 | | | | | ALPAYPA | | | | |
| RRIRFRVA | 179 | FR~A~PA | SEQ ID | DRB1 | −1.98 | | | −0.05 | EAYRFRVA | 83 | −1.49 | | |
| LPATARV | | | 119 | 0701 | | | | | LPAYPAS | | | | |
| YRDERVAL | 180 | RV~L~AY | SEQ ID | DRB3 | | | −1.99 | 0.99 | AYRFRVAL | 84 | | | −0.77 |
| PAYFIIA | | | 120 | 0101 | | | | | PAYPASL | | | | |
| IKNFALPA | 181 | AL~A~PA | SEQ ID | DRB1 | | | | 0.82 | RFRVALPA | 86 | | −1.68 | |
| YPAANYG | | | 122 | 0801 | | | | | YPASLTD | | | | |
| YTYYLPAY | 182 | LP~Y~AS | SEQ ID | DRB1 | | | | 0.96 | FRVALPAY | 87 | | −1.52 | |
| PASLRPP | | | 123 | 0801 | | | | | PASLTDV | | | | |
| PRKAAYPA | 183 | AY~A~LT | SEQ ID | DRB1 | −2.01 | | | 0.86 | VALPAYPA | 89 | −1.36 | | |
| SLTVYLV | | | 125 | 0701 | | | | | SLTDVSL | | | | |
| RPANFLYR | 184 | FL~R~GS | SEQ ID | DRB1 | −2.04 | | | 0.60 | KGVVFLYR | 92 | −1.55 | | |
| EGSLIPV | | | 128 | 0701 | | | | | EGSARYA | | | | |
| APVYFLYR | 185 | FL-R-GS | SEQ ID | DRB1_ | | −2.05 | | 0.45 | KGVVFLYR | 92 | | −1.56 | |
| EGSGPRV | | | 128 | 0801 | | | | | EGSARYA | | | | |
| TPGELYRE | 186 | LY~E~SA | SEQ ID | DRB1_ | −1.99 | | | 0.63 | GVVFLYRE | 93 | −1.02 | | |
| GSAFYLL | | | 129 | 0701 | | | | | GSARYAF | | | | |
| GTAYREGS | 187 | RE~S~RY | SEQ ID | DRB3_ | | | −2.05 | 0.08 | VFLYREGS | 95 | | | −1.61 |
| ARYLFVP | | | 131 | 0101 | | | | | ARYAFSF | | | | |
| KLRLELFL | 188 | EL~L~DP | SEQ ID | DRB1_ | | −2.01 | | 0.64 | DLNGELFL | 97 | | −1.62 | |
| GDPPLAE | | | 133 | 0801 | | | | | GDPPEKL | | | | |
| RLAKLFLG | 189 | LF~G~PP | SEQ ID | DRB1_ | −2.07 | | | 0.96 | LNGELFLG | 98 | −1.04 | | |
| DPPYGPV | | | 134 | 0701 | | | | | DPPEKLT | | | | |
| PALRLFLG | 190 | LF~G~PP | SEQ ID | DRB1_ | | −1.99 | | 0.46 | LNGELFLG | 98 | | −1.50 | |
| DPPPRDV | | | 134 | 0801 | | | | | DPPEKLT | | | | |
| PPINFLGD | 191 | FL~D~PE | SEQ ID | DRB1_ | −1.98 | | | 0.88 | NGELFLGD | 99 | −0.94 | | |
| PPEFGYA | | | 135 | 0701 | | | | | PPEKLTL | | | | |
| FLRYFLGD | 192 | FL~D~PE | SEQ ID | DRB1_ | | −2.03 | | 0.89 | NGELFLGD | 99 | | −1.02 | |
| PPEGRYL | | | 135 | 0801 | | | | | PPEKLTL | | | | |
| NREYFLGD | 193 | FL~D~PE | SEQ ID | DRB3_ | | | −2.01 | 0.69 | NGELFLGD | 99 | | | −0.77 |
| PPEVIIV | | | 135 | 0101 | | | | | PPEKLTL | | | | |

### Example 5: B Cell Epitopes in Brevican

An alternative approach to targeting glioma cells expressing brevican is through targeting of B cell epitopes. This can be achieved either by stimulating antibodies in vivo by vaccination with epitopes designed to induce specific antibodies or by utilization of the variable regions of antibodies to engineer CAR-T cells thus targeted to the B cell epitope bearing cells. In this regard the specificity of upregulation of brevican expression in glioma cells, and its relationship to tumor progression is a critical factor in differentiating between tumor and normal cells. As noted in FIGS. 2 and 3, the B cell epitopes in brevican are primarily located in the region between amino acids 350-650. Two less dominant linear B cell epitopes occur at the N terminal end of the molecule. The B cell epitopes of highest probability of eliciting an antibody response are located either side of the cleavage site prior to the serine 401. We show below in Table 7 the sequences for the B cell epitopes in the N terminal portion following cleavage (SEQs 194-196). These may be administered with MHC IT binding sequences shown in Tables 3 and 5, either as separate peptides or as peptides extended to comprise both B and T cell epitope sequences. Table 8 shows those B cell epitopes located on the C terminal side of the cleavage site (SEQ IDs 220-226). The minimum size of peptide which can locate within an immunoglobulin complementarity determining regions is a pentamer [37]. Antibodies binding peptides centered on any of the pentamers shown in Tables 8 and 10 may be used to target brevican.

Chimeric antigen receptor T cells (CAR-T) have been successfully employed to target hematopoietic cancers, where a broadly conserved set of epitopes may be targeted, such as CD19 with long term control of progression [38, 39]. Targeting solid tumors with CAR T has been more challenging given the diversity of tumor specific targets [28]. Given the relationship of brevican upregulation to the progression of glioma and glioblastoma, brevican and its B cell epitopes provides a broadly applicable target for CAR-T.

TABLE 7
B cell epitope
peptides in N terminal component

| SEQ ID NO: | B cell epitope |
|---|---|
| 194 | LEGDSSEDRAFRVRI |
| 195 | RPPPSRRAVAVLGSP |
| 196 | SAIPEASNPASDGLEAIVT |

TABLE 8
B cell Epitope Core
pentamers in N terminal component

| N terminal | B cell |
|---|---|
| Pentamer | epitope core |
| SEQ ID NO: | pentamer |
| 197 | LEGDS |
| 198 | EGDSS |
| 199 | GDSSE |
| 200 | DSSED |
| 201 | SSEDR |
| 202 | RPPPS |
| 203 | PPPSR |
| 204 | PPSRR |
| 205 | PSAIP |
| 206 | SAIPE |
| 207 | AIPEA |
| 208 | IPEAS |
| 209 | PEASN |
| 210 | EASNP |
| 211 | ASNPA |
| 212 | SNPAS |
| 213 | NPASN |
| 214 | PASNP |
| 215 | ASNPA |
| 216 | SNPAS |
| 217 | NPASD |
| 218 | PASDG |

TABLE 9
B cell epitope peptides in C terminal component

| | |
|---|---|
| SEQ ID NO: 220 | GSSTPEDPAEAPRTLLEFETQSMVPP |
| SEQ ID NO: 221 | EDEEEKEEEEEEEEVEDEALWAWPSELSSP |
| SEQ ID NO: 222 | SSPGPEASLPTEPA |
| SEQ ID NO: 223 | ASPLPDGESEASRPPRVHGPPT |
| SEQ ID NO: 224 | ASPSPSTLVEAREV |
| SEQ ID NO: 225 | RGESEETGSSEGAPSLLPATR |
| SEQ ID NO: 226 | APSEDNSGRTAPAGTSVQAQPVL |

TABLE 10
B cell epitope core
pentamers in C terminal component

| | B cell |
|---|---|
| SEQ | epitope core |
| ID NO: | pentamers |
| 227 | GSSTP |
| 228 | SSTPE |
| 229 | STPED |
| 230 | TPEDP |
| 231 | PEDPA |
| 232 | EDPAE |
| 233 | DPAEA |
| 234 | PAEAP |
| 235 | AEAPR |
| 236 | EAPRT |
| 237 | APRTL |
| 238 | PRTLL |
| 239 | RTLLE |
| 240 | TLLEF |
| 241 | LLEFE |
| 242 | LEFET |
| 243 | EDEEE |
| 244 | DEEEK |
| 245 | EEEKE |
| 246 | EEKEE |
| 247 | EKEEE |
| 248 | KEEEE |
| 249 | EEEEE |
| 250 | EEEEV |
| 251 | EEEVE |
| 252 | EEVED |
| 253 | EVEDE |
| 254 | VEDEA |
| 255 | EDEAL |
| 256 | DEALW |
| 257 | EALWA |
| 258 | ALWAW |
| 259 | LWAWP |
| 260 | SSPGP |
| 261 | SPGPE |
| 262 | PGPEA |
| 263 | GPEAS |
| 264 | ASPLP |
| 265 | SPLPD |
| 266 | PLPDG |
| 267 | LPDGE |
| 268 | PDGES |
| 269 | DGESE |
| 270 | GESEA |
| 271 | ESEAS |
| 272 | SEASR |
| 273 | EASRP |
| 274 | ASRPP |
| 275 | SRPPR |
| 276 | NLASP |
| 277 | LASPS |
| 278 | ASPSP |
| 279 | SPSPS |
| 280 | PSPST |
| 281 | SPSTL |
| 282 | RGESE |
| 283 | GESEE |
| 284 | ESEET |
| 285 | SEETG |
| 286 | EETGS |
| 287 | ETGSS |
| 288 | TGSSE |
| 289 | GSSEG |
| 290 | SSEGA |
| 291 | SEGAP |
| 292 | EGAPS |
| 293 | APSED |
| 294 | PSEDN |
| 295 | SEDNS |
| 296 | EDNSG |
| 297 | DNSGR |
| 298 | NSGRT |
| 299 | SGRTA |
| 300 | GRTAP |
| 301 | RTAPA |
| 302 | TAPAG |
| 303 | APAGT |
| 304 | PAGTS |
| 305 | AGTSV |

### Example 6: Selection of Peptides with Desired Characteristics for Formulation

The method for prediction of MHC binding applied has been described in detail elsewhere (See, e.g., U.S. Pat. No. 10,706,955 incorporated herein by reference in its entirety). Briefly, each amino acid is described by multiple principal components (PC) derived by eigen decomposition and principal component analysis of the correlation matrices between 31 amino acid physical properties derived from experimental studies. PC1 is strongly influenced by the polarity of the amino acid [40, 41]. Thus, to arrive at an index of the polarity of each peptide the average of the PC1 of the constituent amino acids is used. The PC of each amino acid are shown in Table 11.

TABLE 11
Parameters of amino acids

| | Amino | AA | | | Log | | | |
|---|---|---|---|---|---|---|---|---|
| AA name & code | acid | MW | Log P | pl | D7.4 | PC1 | PC2 | PC3 |
| Phenylalanine (Phe) | F | 165.19 | −1.63 | 5.48 | 1.16 | 7.19 | −1.53 | 0.05 |
| Isoleucine (Ile) | I | 131.18 | −1.72 | 6.20 | 0.69 | 6.65 | 0.29 | 0.04 |
| Leucine (Leu) | L | 131.18 | −1.61 | 5.98 | 0.80 | 6.59 | −0.20 | 1.17 |
| Tryptophan (Trp) | W | 204.23 | −1.75 | 5.89 | 1.46 | 5.68 | −3.50 | 0.16 |
| Valine (Val) | V | 117.15 | −2.08 | 5.96 | 0.32 | 4.79 | 1.98 | −0.35 |
| Methionine (Met) | M | 149.21 | −1.84 | 5.74 | 0.51 | 4.14 | −0.43 | −1.46 |
| Tyrosine (Tyr) | Y | 181.19 | −2.42 | 5.66 | 0.55 | 2.58 | −2.06 | 0.37 |
| Cysteine (Cys) | C | 121.16 | −2.49 | 5.07 | 0.82 | 2.11 | 2.74 | −3.84 |
| Alanine (Ala) | A | 89.09 | −2.89 | 6.00 | −0.27 | 0.72 | 2.48 | 1.42 |
| Proline (Pro) | P | 115.13 | −2.50 | 6.30 | 0.15 | −0.03 | −0.36 | 1.87 |
| Glycine (Gly) | G | 75.07 | −3.25 | 5.97 | −0.22 | −0.76 | 3.08 | 1.21 |
| Threonine (Thr) | T | 119.12 | −2.92 | 5.60 | −0.26 | −1.43 | 0.80 | 0.94 |
| Histidine (His) | H | 155.16 | −3.56 | 7.59 | −0.44 | −2.55 | −1.00 | −1.94 |
| Serine (Ser) | S | 105.09 | −3.30 | 5.68 | −0.45 | −2.65 | 1.84 | 1.30 |
| Glutamine (Gln) | Q | 146.15 | −3.24 | 5.65 | −1.00 | −3.97 | −0.47 | 0.15 |
| Asparagine (Asn) | N | 132.12 | −3.41 | 5.41 | −0.98 | −4.35 | 0.22 | 0.30 |
| Glutamic Acid (Glu) | E | 147.13 | −2.94 | 3.22 | −2.19 | −5.70 | 0.34 | −1.46 |
| Aspartic Acid (Asp) | D | 133.10 | −3.38 | 2.77 | −2.06 | −6.04 | 0.03 | −0.18 |
| Arginine (Arg) | R | 174.20 | −4.20 | 10.76 | −1.65 | −6.30 | −2.93 | −0.91 |
| Lysine (Lys) | K | 146.19 | −4.44 | 9.74 | −2.27 | −6.68 | −1.32 | 1.16 |

Table 11 also lists the log P for the octanol:water partition coefficient. The peptide log P is determined for each individual amino acid log Ps divided by the number of amino acids in the peptide. Overall, the average log P of a 9mer peptide (as shown in Table 11) has a value of −2.78, which is equivalent to <0.1% distribution in octanol and 99.9% in water. Peptides with a log Pin excess of −2 is equivalent to approximately 1% in octanol and 99% in water.

Calculation of the above parameters for the peptides generated by variation of the flanking region amino acids allows for selection of a peptide that not only binds the HLA with a desired affinity but is suitable for manufacturing and delivery in a desired formulation.
1. Ostrom Q T, Cote D J, Ascha M, Kruchko C, Bamholtz-Sloan J S. Adult Glioma Incidence and Survival by Race or Ethnicity in the United States From 2000 to 2014. JAMA Oncol. 2018; 4(9):1254-62. Epub 2018/06/23. doi: 10.1001/jamaoncol.2018.1789. PubMed PMID: 29931168; PubMed Central PMCID: PMCPMC6143018.
2. Lefranc M P, Giudicelli V, Ginestoux C, Jabado-Michaloud J, Folch G, Bellahcene F, et al. IMGT, the international ImMunoGeneTics information system. Nucleic acids research. 2009; 37(Database issue):D1006-12. Epub 2008/11/04. doi: 10.1093/nar/gkn838. PubMed PMID: 18978023; PubMed Central PMCID: PMC2686541.
3. Giamanco K A, Matthews R T. The Role of BEHAB/Brevican in the Tumor Microenvironment: Mediating Glioma Cell Invasion and Motility. Adv Exp Med Biol. 2020; 1272:117-32. Epub 2020/08/28. doi: 10.1007/978-3-030-48457-6_7. PubMed PMID: 32845505.
4. Jaworski D M, Kelly G M, Piepmeier J M, Hockfield S. BEHAB (brain enriched hyaluronan binding) is expressed in surgical samples of glioma and in intracranial grafts of invasive glioma cell lines. Cancer Res. 1996; 56(10):2293-8. Epub 1996/05/15. PubMed PMID: 8625302.
5. Jonesco D S, Karsdal M A, Henriksen K. The CNS-specific proteoglycan, brevican, and its ADAMTS4-cleaved fragment show differential serological levels in Alzheimer's disease, other types of dementia and non-demented controls: A cross-sectional study. PloS one. 2020; 15(6):e0234632. Epub 2020/06/20. doi: 10.1371/journal.pone.0234632. PubMed PMID: 32559242; PubMed Central PMCID.
6. Gary S C, Zerillo C A, Chiang V L, Gaw J U, Gray G, Hockfield S. cDNA cloning, chromosomal localization, and expression analysis of human BEHAB/brevican, a brain specific proteoglycan regulated during cortical development and in glioma. Gene. 2000; 256(1-2):139-47. Epub 2000/10/31. doi: 10.1016/s0378-1119(00)00362-0. PubMed PMID: 11054543.
7. Gary S C, Kelly G M, Hockfield S. BEHAB/brevican: a brain-specific lectican implicated in gliomas and glial cell motility. Current opinion in neurobiology. 1998; 8(5):576-81. Epub 1998/11/13. doi: 10.1016/s0959-4388(98)80083-4. PubMed PMID: 9811619.
8. Viapiano M S, Bi W L, Piepmeier J, Hockfield S, Matthews R T. Novel tumor-specific isoforms of BEHAB/brevican identified in human malignant gliomas. Cancer Res. 2005; 65(15):6726-33. Epub 2005/08/03. doi: 10.1158/0008-5472.CAN-05-0585. PubMed PMID: 16061654.
9. Viapiano M S, Matthews R T, Hockfield S. A novel membrane-associated glycovariant of BEHAB/brevican is up-regulated during rat brain development and in a rat model of invasive glioma. J Biol Chem. 2003; 278(35):33239-47. Epub 2003/06/12. doi: 10.1074/jbc.M303480200. PubMed PMID: 12799382.
10. Dwyer C A, Bi W L, Viapiano M S, Matthews R T. Brevican knockdown reduces late-stage glioma tumor aggressiveness. J Neurooncol. 2014; 120(1):63-72. Epub 2014/07/24. doi: 10.1007/s11060-014-1541-z. PubMed PMID: 25052349; PubMed Central PMCID: PMCPMC4177969.
11. Hu B, Kong L L, Matthews R T, Viapiano M S. The proteoglycan brevican binds to fibronectin after proteolytic cleavage and promotes glioma cell motility. J Biol Chem. 2008; 283(36):24848-59. Epub 2008/07/10. doi: 10.1074/jbc.M801433200. PubMed PMID: 18611854; PubMed Central PMCID: PMCPMC3259830.
12. Matthews R T, Gary S C, Zerillo C, Pratta M, Solomon K, Amer E C, et al. Brain-enriched hyaluronan binding (BEHAB)/brevican cleavage in a glioma cell line is mediated by a disintegrin and metalloproteinase with thrombospondin motifs (ADAMTS) family member. J Biol Chem. 2000; 275(30):22695-703. Epub 2000/05/10. doi: 10.1074/jbc.M909764199. PubMed PMID: 10801887.
13. Lendvai D, Morawski M, Negyessy L, Gati G, Jager C, Baksa G, et al. Neurochemical mapping of the human hippocampus reveals perisynaptic matrix around functional synapses in Alzheimer's disease. ActaNeuropathol. 2013; 125(2):215-29. Epub 2012/09/11. doi: 10.1007/s00401-012-1042-0. PubMed PMID: 22961619; PubMed Central PMCID: PMCPMC6485544.
14. Brakebusch C, Seidenbecher C I, Asztely F, Rauch U, Matthies H, Meyer H, et al. Brevican-deficient mice display impaired hippocampal CA1 long-term potentiation but show no obvious deficits in learning and memory. Mol Cell Biol. 2002; 22(21):7417-27. Epub 2002/10/09. doi: 10.1128/MCB.22.21.7417-7427.2002. PubMed PMID: 12370289; PubMed Central PMCID: PMCPMC135663.
15. Zhou X H, Brakebusch C, Matthies H, Oohashi T, Hirsch E, Moser M, et al. Neurocan is dispensable for brain development. Mol Cell Biol. 2001; 21(17):5970-8. Epub 2001/08/04. doi: 10.1128/MCB.21.17.5970-5978.2001. PubMed PMID: 11486035; PubMed Central PMCID: PMCPMC87315.
16. Ogawa T, Hagihara K, Suzuki M, Yamaguchi Y. Brevican in the developing hippocampal fimbria: differential expression in myelinating oligodendrocytes and adult astrocytes suggests a dual role for brevican in central nervous system fiber tract development. The Journal of comparative neurology. 2001; 432(3):285-95. Epub 2001/03/14. doi: 10.1002/cne.1103. PubMed PMID: 11246208.
17. Bekku Y, Rauch U, Ninomiya Y, Oohashi T. Brevican distinctively assembles extracellular components at the large diameter nodes of Ranvier in the CNS. Journal of neurochemistry. 2009; 108(5):1266-76. Epub 2009/01/15. doi: 10.1111/j.1471-4159.2009.05873.x. PubMed PMID: 19141078.
18. Jones L L, Margolis R U, Tuszynski M H. The chondroitin sulfate proteoglycans neurocan, brevican, phosphacan, and versican are differentially regulated following spinal cord injury. Exp Neurol. 2003; 182(2):399-411. Epub 2003/08/05. doi: 10.1016/s0014-4886(03)00087-6. PubMed PMID: 12895450.
19. Pantazopoulos H, Katsel P, Haroutunian V, Chelini G, Klengel T, Berretta S. Molecular signature of extracellular matrix pathology in schizophrenia. The European journal of neuroscience. 2021; 53(12):3960-87. Epub 2020/10/19. doi: 10.1111/cjn.15009. PubMed PMID: 33070392; PubMed Central PMCID: PMCPMC8359380.
20. Lu R, Wu C, Guo L, Liu Y, Mo W, Wang H, et al. The role of brevican in glioma: promoting tumor cell motility in vitro and in vivo. BMC cancer. 2012; 12:607. Epub 2012/12/21. doi: 10.1186/1471-2407-12-607. PubMed PMID: 23253190; PubMed Central PMCID: PMCPMC3575301.
21. Zhang H, Kelly G, Zerillo C, Jaworski D M, Hockfield S. Expression of a cleaved brain-specific extracellular matrix protein mediates glioma cell invasion In vivo. The Journal of neuroscience: the official journal of the Society for Neuroscience. 1998; 18(7):2370-6. Epub 1998/04/16. PubMed PMID: 9502798; PubMed Central PMCID: PMCPMC6793111.
22. So J S, Kim H, Han K S. Mechanisms of Invasion in Glioblastoma: Extracellular Matrix, Ca(2+) Signaling, and Glutamate. Front Cell Neurosci. 2021; 15:663092. Epub 2021/06/22. doi: 10.3389/fncel.2021.663092. PubMed PMID: 34149360; PubMed Central PMCID: PMCPMC8206529.
23. Chen D S, Mellman I. Elements of cancer immunity and the cancer-immune set point. Nature. 2017; 541(7637):321-30. Epub 2017/01/20. doi: 10.1038/nature21349. PubMed PMID: 28102259.
24. McLane L M, Abdel-Hakeem M S, Wherry E J. CD8+ T Cell Exhaustion During Chronic Viral Infection and Cancer. Annu Rev Immunol. 2019; 37:457-95. Epub 2019/01/25. doi: 10.1146/annurev-immunol-041015-055318. PubMed PMID: 30676822.
25. Clancy-Thompson E, Devlin C A, Tyler P M, Servos M M, Ali L R, Ventre K S, et al. Altered Binding of Tumor Antigenic Peptides to MHC Class I Affects CD8(+) T Cell-Effector Responses. Cancer immunology research. 2018; 6(12):1524-36. Epub 2018/10/26. doi: 10.1158/2326-6066.CIR-18-0348. PubMed PMID: 30352798; PubMed Central PMCID: PMCPMC6290996.
26. Riquelme E, Carreno L J, Gonzalez P A, Kalergis A M. The duration of TCR/pMHC interactions regulates CTL effector function and tumor-killing capacity. European journal of immunology. 2009; 39(8):2259-69. Epub 2009/07/29. doi: 10.1002/eji.200939341. PubMed PMID: 19637198.
27. Rani S, Rana R, Saraogi G K, Kumar V, Gupta U. Self-Emulsifying Oral Lipid Drug Delivery Systems: Advances and Challenges. AAPS PharmSciTech. 2019; 20(3):129. Epub 2019/03/01. doi: 10.1208/s12249-019-1335-x. PubMed PMID: 30815765.
28. Mao R, Hussein M S, He Y. Chimeric antigen receptor engineered T cells and their application in the immunotherapy of solid tumours. Expert Rev Mol Med. 2022; 24:e7. Epub 2022/01/29. doi: 10.1017/erm.2021.32. PubMed PMID: 35086597.
29. Maggs L, Cattaneo G, Dal A E, Moghaddam A S, Ferrone S. CAR T Cell-Based Immunotherapy for the Treatment of Glioblastoma. Front Neurosci. 2021; 15:662064. Epub 2021/06/12. doi: 10.3389/fnins.2021.662064. PubMed PMID: 34113233; PubMed Central PMCID: PMCPMC8185049.
30. Chokshi C R, Brakel B A, Tatari N, Savage N, Salim S K, Venugopal C, et al. Advances in Immunotherapy for Adult Glioblastoma. Cancers (Basel). 2021; 13(14). Epub 2021/07/25. doi: 10.3390/cancers13143400. PubMed PMID: 34298615; PubMed Central PMCID: PMCPMC8305609.
31. Geldres C, Savoldo B, Hoyos V, Caruana I, Zhang M, Yvon E, et al. T lymphocytes redirected against the chondroitin sulfate proteoglycan-4 control the growth of multiple solid tumors both in vitro and in vivo. Clin Cancer Res. 2014; 20(4):962-71. Epub 2013/12/18. doi: 10.1158/1078-0432.CCR-13-2218. PubMed PMID: 24334762; PubMed Central PMCID: PMCPMC3944408.
32. Manfredi F, Cianciotti B C, Potenza A, Tassi E, Noviello M, Biondi A, et al. TCR Redirected T Cells for Cancer Treatment: Achievements, Hurdles, and Goals. Frontiers in immunology. 2020; 11:1689. Epub 2020/10/06. doi: 10.3389/fimmu.2020.01689. PubMed PMID: 33013822; PubMed Central PMCID: PMCPMC7494743.
33. Jain D, Mahammad S S, Singh P P, Kodipyaka R. A review on parenteral delivery of peptides and proteins. Drug Dev Ind Pharm. 2019; 45(9):1403-20. Epub 2019/06/20. doi: 10.1080/03639045.2019.1628770. PubMed PMID: 31215293.
34. Badurdeen S, Valladares D B, Farrar J, Gozzer E, Kroeger A, Kuswara N, et al. Sharing experiences: towards an evidence based model of dengue surveillance and outbreak response in Latin America and Asia. BMC public health. 2013; 13:607. doi: 10.1186/1471-2458-13-607. PubMed PMID: 23800243; PubMed Central PMCID: PMC3697990.
35. Hilf N, Kuttruff-Coqui S, Frenzel K, Bukur V, Stevanovic S, Gouttefangeas C, et al. Actively personalized vaccination trial for newly diagnosed glioblastoma. Nature. 2019; 565(7738):240-5. Epub 2018/12/21. doi: 10.1038/s41586-018-0810-y. PubMed PMID: 30568303.
36. Reed S G, Orr M T, Fox C B. Key roles of adjuvants in modern vaccines. Nat Med. 2013; 19(12):1597-608. Epub 2013/12/07. doi: 10.1038/nm.3409. PubMed PMID: 24309663.
37. Robin G, Sato Y, Desplancq D, Rochel N, Weiss E, Martineau P. Restricted diversity of antigen binding residues of antibodies revealed by computational alanine scanning of 227 antibody-antigen complexes. J Mol Biol. 2014; 426(22):3729-43. Epub 2014/09/02. doi: 10.1016/j.jmb.2014.08.013. PubMed PMID: 25174334.
38. Fraietta J A, Lacey S F, Orlando E J, Pruteanu-Malinici I, Gohil M, Lundh S, et al. Determinants of response and resistance to CD19 chimeric antigen receptor (CAR) T cell therapy of chronic lymphocytic leukemia. Nat Med. 2018; 24(5):563-71. Epub 2018/05/02. doi: 10.1038/s41591-018-0010-1. PubMed PMID: 29713085; PubMed Central PMCID: PMCPMC6117613.
39. Melenhorst J J, Chen G M, Wang M, Porter D L, Chen C, Collins M A, et al. Decade-long leukaemia remissions with persistence of CD4(+) CAR T cells. Nature. 2022. Epub 2022/02/04. doi: 10.1038/s41586-021-04390-6. PubMed PMID: 35110735.
40. Bremel R D, Homan E J. An integrated approach to epitope analysis I I: A system for proteomic-scale prediction of immunological characteristics. ImmunomeRes. 2010; 6(1):8. doi: 1745-7580-6-8 [pii]; 10.1186/1745-7580-6-8 [doi].
41. Bremel R D, Homan E J. An integrated approach to epitope analysis I: Dimensional reduction, visualization and prediction of MHC binding using amino acid principal components and regression approaches. Immunome research. 2010; 6:7. Epub 2010/11/04. doi: 10.1186/1745-7580-6-7. PubMed PMID: 21044289; PubMed Central PMCID: PMC2990731.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

We claim:

1. A method for treating a subject with a brain tumor by designing a group of one or more T-cell epitope peptides, or nucleic acids encoding T cell epitope peptides, which have a desired predicted binding affinity for MHC alleles of the subject, comprising the following steps:
identifying a protein of interest in the subject's brain tumor that is not mutated but that is encoded by a gene present at an increased copy number, or the expression of said protein of interest is upregulated;
obtaining the sequence for said protein of interest and identifying a peptide comprising one or more epitopes of interest that is predicted to induce a T cell response to cells of said tumor;
determining T cell exposed motifs in said epitope or epitopes of interest;
determining the predicted binding affinity to the subject's MHC alleles of peptides which comprise each said T cell exposed motif, or a subset thereof;
selecting a group of one or more selected peptides comprising one or more the said T cell exposed motifs and which have a desired predicted binding affinity for one or more of the subject's MHC alleles;
synthesizing said group of one or more selected peptides, or nucleic acids encoding the selected peptides; and
administering the group of selected peptides to the subject.

2. A method for treating a subject with a brain tumor by designing a group of one or more T-cell epitope peptides, or nucleic acids encoding T cell epitope peptides, which have a desired predicted binding affinity for MHC alleles of the subject, comprising the following steps:
identifying a protein of interest in the subject's brain tumor that is not mutated but that is encoded by a gene present at an increased copy number, or the expression of said protein of interest is upregulated;
obtaining the sequence for said protein of interest and identifying a peptide comprising one or more epitopes of interest that is predicted to induce a T cell response to cells of said tumor;
determining T cell exposed motifs in said epitope or epitopes of interest;
determining the predicted binding affinity to the subject's MHC alleles of peptides which comprise each said T cell exposed motif, or a subset thereof;
generating an array of alternative peptides not present in the natural protein sequence, wherein each peptide in the array comprises the amino acids of one of said T cell exposed motifs, and in which one or more of the amino acids not within the T cell exposed motif are substituted to change the predicted MHC binding affinity;
selecting a group of one or more selected peptides from said array of alternative peptides which have a desired predicted binding affinity for one or more of the subject's MHC alleles;
synthesizing said group of one or more selected peptides, or nucleic acids encoding the selected peptides; and
administering the selected peptides to the subject.

3. The method of any one of claims 1 to 2, wherein said brain tumor is of glial cell origin and drawn from the group comprising glioma, glioblastoma, astrocytoma, ependymoma and oligodendrocytoma.

4. The method of any one of claims 1 to 3, wherein said protein forms a component of the extracellular matrix of the tumor.

5. The method of any one of claims 1 to 4, wherein said protein is a proteoglycan from the group comprising brevican, neurocan, versican, and aggrecan.

6. The method of any one of claims 1 to 5, wherein the T cell exposed motifs are brevican T cell exposed motifs derived from amino acids 1 to 400 of SEQ ID NO:1.

7. The method of any one of claims 1 to 6, wherein said T cell epitope is a peptide binding to an MHC I molecule.

8. The method of claim 7 wherein said peptide is from 8-10 amino acids long.

9. The method of any one of claims 1 to 6, wherein said T cell epitope is a peptide binding an MHC II molecule.

10. The method of claim 9 wherein said peptide is from 11-22 amino acids long.

11. The methods of any one of claims 1 to 10, wherein the selected peptides comprise one or more peptides binding an MHC I molecule and one or more peptides binding a MHC II molecule.

12. The method of any one of claims 1 to 11, wherein the binding affinity to an MHC allele is from 200 nM to 1000 nM.

13. The method of any one of claims 1 to 11, wherein the binding affinity to an MHC allele is <200 nm.

14. The method of any one of claims 1 to 11, wherein the binding affinity to an MHC allele is <100 nm.

15. The method of any one of claims 1 to 11, wherein the binding affinity to an MHC allele is <50 nm.

16. The method of any one of claims 1 to 11, wherein said a T cell exposed motif is selected from the group consisting of SEQ ID NOs: 42-75 and combinations thereof.

17. The method of any one of claims 1 to 11, wherein said T cell exposed motif is selected from the group consisting of SEQ ID NOs: 112-147 and combinations thereof.

18. The method of any one of claims 1 and 3 to 11, wherein said selected peptide is from brevican and comprises any sequential 8-10 amino acid peptide from a sequence selected from the group consisting of SEQ ID NOs:2-7.

19. The method of claim 18, wherein said selected peptide(s) is/are selected from the group consisting of from the group SEQ ID NOs: 8-41 and combinations thereof.

20. The method of any one of claims 1 and 3 to 11, wherein said selected peptide(s) is/are from brevican and comprises any sequential 11-22 amino acid peptide from a sequence selected from the group consisting of SEQ ID NOs: 2-7.

21. The method of and one of claims 2 to 11, wherein said selected peptide(s) is/are selected from the group consisting of SEQ ID NOs: 76-111 and combinations thereof.

22. The method of any one of claims 2 to 11, wherein said selected peptide(s) is/are from brevican and comprises a sequence selected from the group consisting of SEQ ID NOs: 148-173 and combinations thereof.

23. The method of any one of claims 2 to 11, wherein said selected peptide(s) is/are from brevican and comprises a sequence from the group consisting of SEQ ID NOs: 174-193 and combinations thereof.

24. The method of any one of claims 1 to 23, wherein the normalized mRNA transcript expression of said protein of interest is in the highest 15% of expression in the tumor proteome.

25. The method of any one of claims 1 to 24, wherein the normalized mRNA transcript expression of said protein of interest is in the highest 2.5% of expression in the tumor proteome.

26. The method of any one of claims 1 to 25, wherein the selected peptides for a subject with HLA A0201 do not comprise any of the peptides encoded by SEQ ID NOs: 306-325 or SEQ ID NOs 329-330.

27. The method of any one of claims 1 to 25, wherein the selected peptides for a subject with HLA A2402 do not comprise any of the peptides encoded by SEQ ID NOs: 326-328.

28. A method for treating a subject with a brain tumor, comprising:
designing a group of one or more B-cell epitope peptides, or nucleic acids encoding B cell epitope peptides, comprising the following steps:
identifying a protein of interest in the subject's brain tumor that is not mutated but that is encoded by a gene present at an increased copy number, or the expression of said protein of interest is upregulated;
obtaining the sequence for said protein of interest and identifying a peptide comprising one or more linear B cell epitopes of interest; and
synthesizing said group of one or more selected peptides, or nucleic acids encoding the selected peptides; and
administering to the subject with a brain tumor.

29. The method of claim 28, wherein said protein of interest is brevican.

30. The method of claim 29, wherein said selected peptides are brevican peptides derived from SEQ ID NO:1.

31. The method of any one of claims 28 to 30, further comprising administering one or more selected peptides identified in any of claims 1 to 25 and or the nucleic acids encoding said peptides.

32. The method of any one of claims 28 to 31, wherein said B cell epitope is selected from the group consisting of SEQ ID NOs.:194-196 and combinations thereof.

33. The method of any one of claims 28 to 31, wherein said B cell epitope is selected from the group consisting of SEQ ID NOs: 220-226 and combinations thereof.

34. The method of any one of claims 28 to 31, wherein said B cell epitope core pentamer comprises a sequence selected from the group consisting of any of SEQ IDs 197-219.

35. The method of any one of claims 28 to 31, wherein said B cell epitope core pentamer comprises a sequence selected from the group consisting of any of SEQ IDs 227-305.

36. An antigen binding molecule elicited by immunization of a subject with a B cell epitope selected from the group consisting of SEQ ID NOs 194-196 and 220-226.

37. An antigen binding molecule elicited by immunization of a subject with a B cell epitope core pentamers selected from the group consisting of SEQ IDs 197-219 and 227-305.

38. The antigen binding molecule of any one of claims 36 to 37, wherein said subject is a human or a non-human subject.

39. T antigen binding molecule elicited by immunization of a subject with a selected peptide identified in any of claims 28 to 38.

40. The antigen binding molecule of claim 39 which comprises a single chain variable fragment derived from an immunoglobulin.

41. The method of any one of claims 1 to 35, further comprising selecting said one or more peptides to provide desired characteristics for formulation and delivery.

42. The method of claim 41 wherein the desired characteristics for formulation and delivery are selected from the group consisting of solubility, stability, and reduced aggregation and combinations thereof.

43. The method of claim 42, wherein the desired characteristic of solubility is achieved by selecting those amino acids not located in the T cell exposed motifs to increase the polarity of the peptide.

44. The method of claim 42, wherein the polarity of the peptide is increased by selecting peptides in which the index of polarity determined by the average of the first principal component of the amino acids in the peptide is less than or equal to 1.

45. The method of claim 42, wherein the polarity of the peptide is increased by selecting peptides in which the index of polarity determined by the average of the first principal component of the amino acids in the peptide is less than or equal to 2.

46. The method of claim 42, wherein the desired characteristic of solubility is achieved by selecting amino acids not located in the T cell exposed motifs to provide an average log P of the peptide for octanol:water of less than or equal to −2.0.

47. The method of claim 42 wherein the desired characteristic of solubility is achieved by selecting amino acids not located within the T cell exposed motif from the group comprising one or more of arginine, lysine, aspartic acid and glutamic acid.

48. The method of claim 42, wherein the desired characteristic of stability is achieved by selecting amino acids not located within the T cell exposed motif to reduce oxidation and deamidation.

49. The method of claim 48, wherein the amino acids not located within the T cell exposed motif are selected to exclude methionine, tryptophan, histidine, cysteine and tyrosine.

50. The method of claim 48 wherein the amino acids not located within the T cell exposed motif are selected to exclude asparagine and glutamine.

51. The method of any one of claims 1 to 35 and 41 to 50, wherein the selected peptides have a molecular weight less than 4000 daltons.

52. The method of any one of claims 1 to 35 and 41 to 50, wherein the selected peptides have a molecular weight of 1500-4000 daltons.

53. The method of any one of claims 1 to 35 and 41 to 50, wherein the selected peptides have a molecular weight less than 1500 daltons.

54. A vaccination regimen comprising:
administering one or more selected peptides or the nucleic acids encoding them, selected according to the method of any one of claims 1 to 35 and 41 to 53 to a subject with a brain tumor.

55. A vaccination regimen comprising:
administering one or more peptides or the nucleic acids encoding them, selected from the group consisting of SEQ ID NOs: 2-193 to bind with a desired affinity to the MHC alleles of a subject diagnosed as having a glioma, and optionally where the subject has not been biopsied prior to said administration.

56. The vaccination regimens of any one of claims 54 to 55, wherein the one or more selected peptides comprise both MHC I and MHC II binding peptides.

57. The vaccination regimen of any one of claims 54 to 55 wherein the one or more selected peptides comprise both MHC binding peptides and one or more B cell epitopes selected from the group consisting of SEQ ID NOs: 194-219.

58. The vaccination regimen of any one of claims 54 to 57, wherein the vaccination is accompanied by administration of an immunotherapy intervention.

59. The method of claim 58, wherein the immunotherapy intervention is a checkpoint inhibitor drug.

60. The vaccination regimen of any one of claims 54 to 59, wherein the vaccine is administered to the subject parenterally.

61. The vaccination regimen of claim 60, wherein the vaccine is administered intradermally, intra muscularly, intratumorally, or subcutaneously.

62. The vaccination regimen of any one of claims 54 to 59, wherein the vaccine is administered to the subject by a non-parenteral route.

63. The vaccination regimen of claim 62, wherein the non-parenteral route is selected from the group consisting of intranasal, pulmonary inhalation, rectal, and oral routes.

64. The vaccination regimen of claim 63, wherein the oral route is selected from the group consisting of buccal, pharyngeal and sublingual routes.

65. The vaccine of claim 63, wherein the oral route is a gastrointestinal route.

66. The vaccination regimen of any one of claims 63 to 65, wherein the vaccine is delivered as a coated tablet.

67. The vaccination regimen of claim 66, wherein the vaccine is delivered as an enteric coated capsule.

68. The vaccination regimen of any one of claims 54 to 67, wherein the peptides are delivered in a lipid drug delivery system selected from the group consisting of lipid nanoparticles, emulsions, self-emulsifying drug delivery systems, nanocapsules and liposomes.

69. The vaccination regimen of any one of claims 54 to 67, wherein the peptides are delivered in a particulate form.

70. The vaccination regimen of any one of claims 5 to 67, wherein the peptides are formulated for delivery via a system selected from the group consisting of a nanoparticle system, a hydrogel system, a mucoadhesive patch, and a microneedle.

71. The vaccination regimen of claim 70, wherein the vaccine is delivered in a microneedle patch.

72. The vaccination regimen of claim 70, wherein the vaccine is delivered by a multi-needle delivery device.

73. The vaccination regimen of any one of claims 54 to 72, wherein the peptides are administered with an adjuvant.

74. The vaccination regimen of any one of claims 54 to 72, wherein the vaccination is preceded by administration of an adjuvant.

75. The vaccination regimen of any one of claims 54 to 74, wherein the peptides are administered with a pharmaceutically acceptable excipient.

76. The vaccination regimen of any one of claims 54 to 75, wherein the peptides are lyophilized.

77. The vaccination regimen of any one of claims 54 to 76, further comprising contacting the peptides with autologous dendritic cells derived from the subject and administering said dendritic cells to the subject of origin.

78. A method of treating a subject with glioma comprising:
engineering T cells to comprise the antigen binding molecule of claims 39-40;
culturing the T cells in vitro; and
administering the T cells by adoptive transfer to the subject affected by a tumor.

79. The method of claim 78 wherein said T cell is allogenic.

80. The method of claim 78 wherein said T cell is autologous.

81. The method of any one of claims 1 to 27, further comprising:
harvesting T cells from the subject and determining clones that are cognate for the T cell epitopes administered to the subject;
expanding the cognate clones in vitro; and
administering the expanded T cell clones to the subject.

82. The method of any one of claims 1 to 27, further comprising:
harvesting T cells from the subject and determining clones that are cognate for the T cell epitopes administered to the subject;
sequencing the T cell receptors of said cognate T cells;
engineering T cells to carry the T cell receptors so determined to be cognate for the T cell epitopes;
expanding the engineered T cells in vitro; and
administering the engineered T cell clones to the subject.

83. The method of claim 82 wherein the engineered T cells are allogenic.

84. The method of claim 82 wherein the engineered T cells are autologous.

85. A method of treatment of a subject clinically diagnosed with a glioma comprising:
determining the HLA alleles of the subject;
selecting an array of peptides from brevican that have a desired binding affinity for one or more HLA carried by the subject; and
administering the array of peptides, or the nucleic acids encoding the peptides to the subject, wherein the administration is prior to a biopsy of the glioma.

86. The method of claim 85, further comprising designing one or more alternative peptides with desired binding affinity to the subject's HLA alleles, comprising:
selecting T cell exposed motifs of interest in brevican;
generating an array of alternative peptides not present in the natural protein sequence, wherein each peptide in the array comprises the amino acids of one of said T cell exposed motifs, and in which one or more of the amino acids not within the T cell exposed motif are substituted to change the predicted MHC binding affinity;
selecting a group of one or more selected peptides from said array of alternative peptides which have a desired predicted binding affinity for one or more of the subject's MHC alleles;
synthesizing said group of one or more selected peptides, or nucleic acids encoding the selected peptides; and
administering the array of peptides, or the nucleic acids encoding the peptides to the subject.

87. The method of any one of claims 85 to 86, wherein the T cell exposed motifs are brevican T cell exposed motifs located within SEQ ID NO: 1.

88. The method of any one of claims 85 to 87, wherein said T cell epitope is a peptide binding to an MHC I molecule.

89. The method of claim 88, wherein said peptide is from 8-10 amino acids long.

90. The method of any one of claims 85 to 89, wherein said T cell epitope is a peptide binding an MHC II molecule.

91. The method of claim 90, wherein said peptide is from 11-22 amino acids long.

92. The method of any one of claims 85 to 91, wherein the selected peptides comprise one or more peptides binding an MHC I molecule and one or more peptides binding a MHC II molecule.

93. The method of any one of claims 85 to 92, wherein the binding affinity to an MHC allele is <200 nm.

94. The method of any one of claims 85 to 92, wherein the binding affinity to an MHC allele is <100 nm.

95. The method of any one of claims 85 to 92, wherein the binding affinity to an MHC allele is <50 nm.

96. The method of any one of claims 85 to 92, wherein the binding affinity to an MHC allele is <20 nm.

97. The method of any of claims 85 to 96, wherein said peptide(s) comprise(s) a T cell exposed motif selected from the group consisting of SEQ ID NOs.: 42-75.

98. The method of claim 1 or claim 2 wherein said peptide(s) comprise(s) a T cell exposed motif selected from the group consisting of SEQ ID NOs: 112-147.

99. The method of any one of claims 85 to 98, wherein said peptide is not in the top 1% highest affinity binding peptides SEQ ID NO: 1.

100. The method of any one of claims 85 to 99, wherein the peptide is not selected to bind to A0201 or A2402.


SEQUENCE LISTING (328 sequences)

SEQ ID NO: 1 - protein, 400 aa, Homo sapiens
MAQLFLPLLAALVLAQAPAALADVLEGDSSEDRAFRVRIAGDAPLQGVLGGALTIPCHVHYLRPPPSRRAVLGSPRVKWTFLSRGREAEVLVARGVRVKVNEAYRFRVALPAYPASLTDVSLALSELRPNDSGIYRCEVQHGIDDSSDAVEVKVKGVVFLYREGSARYAFSFSGAQEACARIGAHIATPEQLYAAYLGGYEQCDAGWLSDQTVRYPIQTPREACYGDMDGFPGVRNYGVVDPDDLYDVYCYAEDLNGELFLGDPPEKLTLEEARAYCQERGAEIATTGQLYAAWDGGLDHCSPGWLADGSVRYPIVTPSQRCGGGLPGVKTLFLFPNQTGFPNKHSRFNVYCFRDSAQPSAIPEASNPASNPASDGLEAIVTVTETLEELQLPQEATESE

SEQ ID NO: 2 - protein, 20 aa, Synthetic construct
GALTIPCHVHYLRPPPSRRA

SEQ ID NO: 3 - protein, 25 aa, Synthetic construct
NEAYRFRVALPAYPASLTDVSLALS

SEQ ID NO: 4 - protein, 18 aa, Synthetic construct
KGVVFLYREGSARYAFSF

SEQ ID NO: 5 - protein, 20 aa, Synthetic construct
ARYAFSFSGAQEACARIGAH

SEQ ID NO: 6 - protein, 30 aa, Synthetic construct
DLNGELFLGDPPEKLTLEEARAYCQERGAE

SEQ ID NO: 7 - protein, 24 aa, Synthetic construct
GLPGVKTLFLFPNQTGFPNKHSRF

SEQ ID NO: 8 - protein, 9 aa, Synthetic construct
GALTIPCHV

SEQ ID NO: 9 - protein, 9 aa, Synthetic construct
ALTIPCHVH

SEQ ID NO: 10 - protein, 9 aa, Synthetic construct
LTIPCHVHY

SEQ ID NO: 11 - protein, 9 aa, Synthetic construct
TIPCHVHYL

SEQ ID NO: 12 - protein, 9 aa, Synthetic construct
IPCHVHYLR

SEQ ID NO: 13 - protein, 9 aa, Synthetic construct
PCHVHYLRP

SEQ ID NO: 14 - protein, 9 aa, Synthetic construct
EAYRFRVAL

SEQ ID NO: 15 - protein, 9 aa, Synthetic construct
AYRFRVALP

SEQ ID NO: 16 - protein, 9 aa, Synthetic construct
YRFRVALPA

SEQ ID NO: 17 - protein, 9 aa, Synthetic construct
RFRVALPAY

SEQ ID NO: 18 - protein, 9 aa, Synthetic construct
FRVALPAYP

SEQ ID NO: 19 - protein, 9 aa, Synthetic construct
RVALPAYPA

SEQ ID NO: 20 - protein, 9 aa, Synthetic construct
ALPAYPASL

SEQ ID NO: 21 - protein, 9 aa, Synthetic construct
GVVFLYREG

SEQ ID NO: 22 - protein, 9 aa, Synthetic construct
VVFLYREGS

SEQ ID NO: 23 - protein, 9 aa, Synthetic construct
VFLYREGSA

SEQ ID NO: 24 - protein, 9 aa, Synthetic construct
ARYAFSFSG

SEQ ID NO: 25 - protein, 9 aa, Synthetic construct
RYAFSFSGA

SEQ ID NO: 26 - protein, 9 aa, Synthetic construct
FSFSGAQEA

SEQ ID NO: 27 - protein, 9 aa, Synthetic construct
SFSGAQEAC

SEQ ID NO: 28 - protein, 9 aa, Synthetic construct
DLNGELFLG

SEQ ID NO: 29 - protein, 9 aa, Synthetic construct
ELFLGDPPE

SEQ ID NO: 30 - protein, 9 aa, Synthetic construct
LFLGDPPEK

SEQ ID NO: 31 - protein, 9 aa, Synthetic construct
FLGDPPEKL

SEQ ID NO: 32 - protein, 9 aa, Synthetic construct
LGDPPEKLT

SEQ ID NO: 33 - protein, 9 aa, Synthetic construct
KLTLEEARA

SEQ ID NO: 34 - protein, 9 aa, Synthetic construct
LTLEEARAY

SEQ ID NO: 35 - protein, 9 aa, Synthetic construct
TLEEARAYC

SEQ ID NO: 36 - protein, 9 aa, Synthetic construct
GLPGVKTLF

SEQ ID NO: 37 - protein, 9 aa, Synthetic construct
LPGVKTLFL

SEQ ID NO: 38 - protein, 9 aa, Synthetic construct
PGVKTLFLF

SEQ ID NO: 39 - protein, 9 aa, Synthetic construct
LFLFPNQTG

SEQ ID NO: 40 - protein, 9 aa, Synthetic construct
FLFPNQTGF

SEQ ID NO: 41 - protein, 9 aa, Synthetic construct
LFPNQTGFP

SEQ ID NO: 42 - protein, 5 aa, Synthetic construct
TIPCH

SEQ ID NO: 43 - protein, 5 aa, Synthetic construct
IPCHV

SEQ ID NO: 44 - protein, 5 aa, Synthetic construct
PCHVH

SEQ ID NO: 45 - protein, 5 aa, Synthetic construct
CHVHY

SEQ ID NO: 46 - protein, 5 aa, Synthetic construct
HVHYL

SEQ ID NO: 47 - protein, 5 aa, Synthetic construct
VHYLR

SEQ ID NO: 48 - protein, 5 aa, Synthetic construct
RFRVA

SEQ ID NO: 49 - protein, 5 aa, Synthetic construct
FRVAL

SEQ ID NO: 50 - protein, 5 aa, Synthetic construct
RVALP

SEQ ID NO: 51 - protein, 5 aa, Synthetic construct
VALPA

SEQ ID NO: 52 - protein, 5 aa, Synthetic construct
ALPAY

SEQ ID NO: 53 - protein, 5 aa, Synthetic construct
LPAYP

SEQ ID NO: 54 - protein, 5 aa, Synthetic construct
AYPAS

SEQ ID NO: 55 - protein, 5 aa, Synthetic construct
FLYRE

SEQ ID NO: 56 - protein, 5 aa, Synthetic construct
LYREG

SEQ ID NO: 57 - protein, 5 aa, Synthetic construct
YREGS

SEQ ID NO: 58 - protein, 5 aa, Synthetic construct
AFSFS

SEQ ID NO: 59 - protein, 5 aa, Synthetic construct
FSFSG

SEQ ID NO: 60 - protein, 5 aa, Synthetic construct
SGAQE

SEQ ID NO: 61 - protein, 5 aa, Synthetic construct
GAQEA

SEQ ID NO: 62 - protein, 5 aa, Synthetic construct
GELFL

SEQ ID NO: 63 - protein, 5 aa, Synthetic construct
LGDPP

SEQ ID NO: 64 - protein, 5 aa, Synthetic construct
GDPPE

SEQ ID NO: 65 - protein, 5 aa, Synthetic construct
DPPEK

SEQ ID NO: 66 - protein, 5 aa, Synthetic construct
PPEKL

SEQ ID NO: 67 - protein, 5 aa, Synthetic construct
LEEAR

SEQ ID NO: 68 - protein, 5 aa, Synthetic construct
EEARA

SEQ ID NO: 69 - protein, 5 aa, Synthetic construct
EARAY

SEQ ID NO: 70 - protein, 5 aa, Synthetic construct
GVKTL

SEQ ID NO: 71 - protein, 5 aa, Synthetic construct
VKTLF

SEQ ID NO: 72 - protein, 5 aa, Synthetic construct
KTLFL

SEQ ID NO: 73 - protein, 5 aa, Synthetic construct
FPNQT

SEQ ID NO: 74 - protein, 5 aa, Synthetic construct
PNQTG

SEQ ID NO: 75 - protein, 5 aa, Synthetic construct
NQTGF

SEQ ID NO: 76 - protein, 15 aa, Synthetic construct
GALTIPCHVHYLRPP

SEQ ID NO: 77 - protein, 15 aa, Synthetic construct
ALTIPCHVHYLRPPP

SEQ ID NO: 78 - protein, 15 aa, Synthetic construct
LTIPCHVHYLRPPPS

SEQ ID NO: 79 - protein, 15 aa, Synthetic construct
TIPCHVHYLRPPPSR

SEQ ID NO: 80 - protein, 15 aa, Synthetic construct
IPCHVHYLRPPPSRR

SEQ ID NO: 81 - protein, 15 aa, Synthetic construct
PCHVHYLRPPPSRRA

SEQ ID NO: 82 - protein, 15 aa, Synthetic construct
NEAYRFRVALPAYPA

SEQ ID NO: 83 - protein, 15 aa, Synthetic construct
EAYRFRVALPAYPAS

SEQ ID NO: 84 - protein, 15 aa, Synthetic construct
AYRFRVALPAYPASL

SEQ ID NO: 85 - protein, 15 aa, Synthetic construct
YRFRVALPAYPASLT

SEQ ID NO: 86 - protein, 15 aa, Synthetic construct
RFRVALPAYPASLTD

SEQ ID NO: 87 - protein, 15 aa, Synthetic construct
FRVALPAYPASLTDV

SEQ ID NO: 88 - protein, 15 aa, Synthetic construct
RVALPAYPASLTDVS

SEQ ID NO: 89 - protein, 15 aa, Synthetic construct
VALPAYPASLTDVSL

SEQ ID NO: 90 - protein, 15 aa, Synthetic construct
ALPAYPASLTDVSLA

SEQ ID NO: 91 - protein, 15 aa, Synthetic construct
LPAYPASLTDVSLAL

SEQ ID NO: 92 - protein, 15 aa, Synthetic construct
KGVVFLYREGSARYA

SEQ ID NO: 93 - protein, 15 aa, Synthetic construct
GVVFLYREGSARYAF

SEQ ID NO: 94 - protein, 15 aa, Synthetic construct
VVFLYREGSARYAFS

SEQ ID NO: 95 - protein, 15 aa, Synthetic construct
VFLYREGSARYAFSF

SEQ ID NO: 96 - protein, 15 aa, Synthetic construct
ARYAFSFSGAQEACA

SEQ ID NO: 97 - protein, 15 aa, Synthetic construct
DLNGELFLGDPPEKL

SEQ ID NO: 98 - protein, 15 aa, Synthetic construct
LNGELFLGDPPEKLT

SEQ ID NO: 99 - protein, 15 aa, Synthetic construct
NGELFLGDPPEKLTL

SEQ ID NO: 100 - protein, 15 aa, Synthetic construct
GELFLGDPPEKLTLE

SEQ ID NO: 101 - protein, 15 aa, Synthetic construct
ELFLGDPPEKLTLEE

SEQ ID NO: 102 - protein, 15 aa, Synthetic construct
PEKLTLEEARAYCQE

SEQ ID NO: 103 - protein, 15 aa, Synthetic construct
GLPGVKTLFLFPNQT

SEQ ID NO: 104 - protein, 15 aa, Synthetic construct
LPGVKTLFLFPNQTG

SEQ ID NO: 105 - protein, 15 aa, Synthetic construct
PGVKTLFLFPNQTGF

SEQ ID NO: 106 - protein, 15 aa, Synthetic construct
GVKTLFLFPNQTGFP

SEQ ID NO: 107 - protein, 15 aa, Synthetic construct
VKTLFLFPNQTGFPN

SEQ ID NO: 108 - protein, 15 aa, Synthetic construct
KTLFLFPNQTGFPNK

SEQ ID NO: 109 - protein, 15 aa, Synthetic construct
TLFLFPNQTGFPNKH

SEQ ID NO: 110 - protein, 15 aa, Synthetic construct
LFLFPNQTGFPNKHS

SEQ ID NO: 111 - protein, 15 aa, Synthetic construct
FLFPNQTGFPNKHSR

SEQ ID NO: 112 - protein, 5 aa, Synthetic construct
IPHHY

SEQ ID NO: 113 - protein, 5 aa, Synthetic construct
PCVYL

SEQ ID NO: 114 - protein, 5 aa, Synthetic construct
CHHLR

SEQ ID NO: 115 - protein, 5 aa, Synthetic construct
HVYRP

SEQ ID NO: 116 - protein, 5 aa, Synthetic construct
VHLPP

SEQ ID NO: 117 - protein, 5 aa, Synthetic construct
HYRPP

SEQ ID NO: 118 - protein, 5 aa, Synthetic construct
RFVLP

SEQ ID NO: 119 - protein, 5 aa, Synthetic construct
FRAPA

SEQ ID NO: 120 - protein, 5 aa, Synthetic construct
RVLAY

SEQ ID NO: 121 - protein, 5 aa, Synthetic construct
VAPYP

SEQ ID NO: 122 - protein, 5 aa, Synthetic construct
ALAPA

SEQ ID NO: 123 - protein, 5 aa, Synthetic construct
LPYAS

SEQ ID NO: 124 - protein, 5 aa, Synthetic construct
PAPSL

SEQ ID NO: 125 - protein, 5 aa, Synthetic construct
AYALT

SEQ ID NO: 126 - protein, 5 aa, Synthetic construct
YPSTD

SEQ ID NO: 127 - protein, 5 aa, Synthetic construct
PALDV

SEQ ID NO: 128 - protein, 5 aa, Synthetic construct
FLRGS

SEQ ID NO: 129 - protein, 5 aa, Synthetic construct
LYESA

SEQ ID NO: 130 - protein, 5 aa, Synthetic construct
YRGAR

SEQ ID NO: 131 - protein, 5 aa, Synthetic construct
RESRY

SEQ ID NO: 132 - protein, 5 aa, Synthetic construct
FSSAQ

SEQ ID NO: 133 - protein, 5 aa, Synthetic construct
ELLDP

SEQ ID NO: 134 - protein, 5 aa, Synthetic construct
LFGPP

SEQ ID NO: 135 - protein, 5 aa, Synthetic construct
FLDPE

SEQ ID NO: 136 - protein, 5 aa, Synthetic construct
LGPEK

SEQ ID NO: 137 - protein, 5 aa, Synthetic construct
GDPKL

SEQ ID NO: 138 - protein, 5 aa, Synthetic construct
TLERA

SEQ ID NO: 139 - protein, 5 aa, Synthetic construct
VKLLF

SEQ ID NO: 140 - protein, 5 aa, Synthetic construct
KTFFP

SEQ ID NO: 141 - protein, 5 aa, Synthetic construct
TLLPN

SEQ ID NO: 142 - protein, 5 aa, Synthetic construct
LFFNQ

SEQ ID NO: 143 - protein, 5 aa, Synthetic construct
FLPQT

SEQ ID NO: 144 - protein, 5 aa, Synthetic construct
LFNTG

SEQ ID NO: 145 - protein, 5 aa, Synthetic construct
FPQGF

SEQ ID NO: 146 - protein, 5 aa, Synthetic construct
PNTFP

SEQ ID NO: 147 - protein, 5 aa, Synthetic construct
NQGPN

SEQ ID NO: 148 - protein, 9 aa, Synthetic construct
GERTIPCHT

SEQ ID NO: 149 - protein, 9 aa, Synthetic construct
DLTIPCHVR

SEQ ID NO: 150 - protein, 9 aa, Synthetic construct
DVRPCHVHF

SEQ ID NO: 151 - protein, 9 aa, Synthetic construct
GAPPCHVHR

SEQ ID NO: 152 - protein, 9 aa, Synthetic construct
LERPCHVHE

SEQ ID NO: 153 - protein, 9 aa, Synthetic construct
VSYCHVHYR

SEQ ID NO: 154 - protein, 9 aa, Synthetic construct
VDPHVHYLR

SEQ ID NO: 155 - protein, 9 aa, Synthetic construct
GEEHVHYLA

SEQ ID NO: 156 - protein, 9 aa, Synthetic construct
EVFRFRVAP

SEQ ID NO: 157 - protein, 9 aa, Synthetic construct
GAFRFRVAR

SEQ ID NO: 158 - protein, 9 aa, Synthetic construct
EDFRFRVAE

SEQ ID NO: 159 - protein, 9 aa, Synthetic construct
TAGVALPAR

SEQ ID NO: 160 - protein, 9 aa, Synthetic construct
DGLVALPAR

SEQ ID NO: 161 - protein, 9 aa, Synthetic construct
PDFVALPAR

SEQ ID NO: 162 - protein, 9 aa, Synthetic construct
AEGVALPAA

SEQ ID NO: 163 - protein, 9 aa, Synthetic construct
DEALPAYPA

SEQ ID NO: 164 - protein, 9 aa, Synthetic construct
REAFLYREP

SEQ ID NO: 165 - protein, 9 aa, Synthetic construct
IAILYREGR

SEQ ID NO: 166 - protein, 9 aa, Synthetic construct
GGTYREGSF

SEQ ID NO: 167 - protein, 9 aa, Synthetic construct
LEFYREGSV

SEQ ID NO: 168 - protein, 9 aa, Synthetic construct
DALSGAQER

SEQ ID NO: 169 - protein, 9 aa, Synthetic construct
REVGAQEAT

SEQ ID NO: 170-171 - skipped (2 entries)

SEQ ID NO: 172 - protein, 9 aa, Synthetic construct
EFALGDPPR

SEQ ID NO: 173 - protein, 9 aa, Synthetic construct
AEGPPEKLE

SEQ ID NO: 174 - protein, 15 aa, Synthetic construct
YVREPCHVHYLALPA

SEQ ID NO: 175 - protein, 15 aa, Synthetic construct
RFIVHVHYLRPDPPA

SEQ ID NO: 176 - protein, 15 aa, Synthetic construct
GKRNVHYLRPPYAVA

SEQ ID NO: 177 - protein, 15 aa, Synthetic construct
AKVLHYLRPPPPPLY

SEQ ID NO: 178 - protein, 15 aa, Synthetic construct
GDYRRFRVALPAAPV

SEQ ID NO: 179 - protein, 15 aa, Synthetic construct
RRIRFRVALPATARV

SEQ ID NO: 180 - protein, 15 aa, Synthetic construct
YRDERVALPAYFIIA

SEQ ID NO: 181 - protein, 15 aa, Synthetic construct
IKNFALPAYPAANYG

SEQ ID NO: 182 - protein, 15 aa, Synthetic construct
YTYYLPAYPASLRPP

SEQ ID NO: 183 - protein, 15 aa, Synthetic construct
PRKAAYPASLTVYLV

SEQ ID NO: 184 - protein, 15 aa, Synthetic construct
RPANFLYREGSLIPV

SEQ ID NO: 185 - protein, 15 aa, Synthetic construct
APVYFLYREGSGPRV

SEQ ID NO: 186 - protein, 15 aa, Synthetic construct
TPGELYREGSAFYLL

SEQ ID NO: 187 - protein, 15 aa, Synthetic construct
GTAYREGSARYLFVP

SEQ ID NO: 188 - protein, 15 aa, Synthetic construct
KLRLELFLGDPPLAE

SEQ ID NO: 189 - protein, 15 aa, Synthetic construct
RLAKLFLGDPPYGPV

SEQ ID NO: 190 - protein, 15 aa, Synthetic construct
PALRLFLGDPPPRDV

SEQ ID NO: 191 - protein, 15 aa, Synthetic construct
PPINFLGDPPEFGYA

SEQ ID NO: 192 - protein, 15 aa, Synthetic construct
FLRYFLGDPPEGRYL

SEQ ID NO: 193 - protein, 15 aa, Synthetic construct
NREYFLGDPPEVIIV

SEQ ID NO: 194 - protein, 15 aa, Synthetic construct
LEGDSSEDRAFRVRI

SEQ ID NO: 195 - protein, 15 aa, Synthetic construct
RPPPSRRAVAVLGSP

SEQ ID NO: 196 - protein, 19 aa, Synthetic construct
SAIPEASNPASDGLEAIVT

SEQ ID NO: 197 - protein, 5 aa, Synthetic construct
LEGDS

SEQ ID NO: 198 - protein, 5 aa, Synthetic construct
EGDSS

SEQ ID NO: 199 - protein, 5 aa, Synthetic construct
GDSSE

SEQ ID NO: 200 - protein, 5 aa, Synthetic construct
DSSED

SEQ ID NO: 201 - protein, 5 aa, Synthetic construct
SSEDR

SEQ ID NO: 202 - protein, 5 aa, Synthetic construct
RPPPS

SEQ ID NO: 203 - protein, 5 aa, Synthetic construct
PPPSR

SEQ ID NO: 204 - protein, 5 aa, Synthetic construct
PPSRR

SEQ ID NO: 205 - protein, 5 aa, Synthetic construct
PSAIP

SEQ ID NO: 206 - protein, 5 aa, Synthetic construct
SAIPE

SEQ ID NO: 207 - protein, 5 aa, Synthetic construct
AIPEA

SEQ ID NO: 208 - protein, 5 aa, Synthetic construct
IPEAS

SEQ ID NO: 209 - protein, 5 aa, Synthetic construct
PEASN

SEQ ID NO: 210 - protein, 5 aa, Synthetic construct
EASNP

SEQ ID NO: 211 - protein, 5 aa, Synthetic construct
ASNPA

SEQ ID NO: 212 - protein, 5 aa, Synthetic construct
SNPAS

SEQ ID NO: 213 - protein, 5 aa, Synthetic construct
NPASN

SEQ ID NO: 214 - protein, 5 aa, Synthetic construct
PASNP

SEQ ID NO: 215 - protein, 5 aa, Synthetic construct
ASNPA

SEQ ID NO: 216 - protein, 5 aa, Synthetic construct
SNPAS

SEQ ID NO: 217 - protein, 5 aa, Synthetic construct
NPASD

SEQ ID NO: 218 - protein, 5 aa, Synthetic construct
PASDG

SEQ ID NO: 219 - protein, 5 aa, Synthetic construct
ASDGL

SEQ ID NO: 220 - protein, 26 aa, Synthetic construct
GSSTPEDPAEAPRTLLEFETQSMVPP

SEQ ID NO: 221 - protein, 30 aa, Synthetic construct
EDEEEKEEEEEEEEVEDEALWAWPSELSSP

SEQ ID NO: 222 - protein, 14 aa, Synthetic construct
SSPGPEASLPTEPA

SEQ ID NO: 223 - protein, 22 aa, Synthetic construct
ASPLPDGESEASRPPRVHGPPT

SEQ ID NO: 224 - protein, 14 aa, Synthetic construct
ASPSPSTLVEAREV

SEQ ID NO: 225 - protein, 21 aa, Synthetic construct
RGESEETGSSEGAPSLLPATR

SEQ ID NO: 226 - protein, 23 aa, Synthetic construct
APSEDNSGRTAPAGTSVQAQPVL

SEQ ID NO: 227 - protein, 5 aa, Synthetic construct
GSSTP

SEQ ID NO: 228 - protein, 5 aa, Synthetic construct
SSTPE

SEQ ID NO: 229 - protein, 5 aa, Synthetic construct
STPED

SEQ ID NO: 230 - protein, 5 aa, Synthetic construct
TPEDP

SEQ ID NO: 231 - protein, 5 aa, Synthetic construct
PEDPA

SEQ ID NO: 232 - protein, 5 aa, Synthetic construct
EDPAE

SEQ ID NO: 233 - protein, 5 aa, Synthetic construct
DPAEA

SEQ ID NO: 234 - protein, 5 aa, Synthetic construct
PAEAP

SEQ ID NO: 235 - protein, 5 aa, Synthetic construct
AEAPR

SEQ ID NO: 236 - protein, 5 aa, Synthetic construct
EAPRT

SEQ ID NO: 237 - protein, 5 aa, Synthetic construct
APRTL

SEQ ID NO: 238 - protein, 5 aa, Synthetic construct
PRTLL

SEQ ID NO: 239 - protein, 5 aa, Synthetic construct
RTLLE

SEQ ID NO: 240 - protein, 5 aa, Synthetic construct
TLLEF

SEQ ID NO: 241 - protein, 5 aa, Synthetic construct
LLEFE

SEQ ID NO: 242 - protein, 5 aa, Synthetic construct
LEFET

SEQ ID NO: 243 - protein, 5 aa, Synthetic construct
EDEEE

SEQ ID NO: 244 - protein, 5 aa, Synthetic construct
DEEEK

SEQ ID NO: 245 - protein, 5 aa, Synthetic construct
EEEKE

SEQ ID NO: 246 - protein, 5 aa, Synthetic construct
EEKEE

SEQ ID NO: 247 - protein, 5 aa, Synthetic construct
EKEEE

SEQ ID NO: 248 - protein, 5 aa, Synthetic construct
KEEEE

SEQ ID NO: 249 - protein, 5 aa, Synthetic construct
EEEEE

SEQ ID NO: 250 - protein, 5 aa, Synthetic construct
EEEEV

SEQ ID NO: 251 - protein, 5 aa, Synthetic construct
EEEVE

SEQ ID NO: 252 - protein, 5 aa, Synthetic construct
EEVED

SEQ ID NO: 253 - protein, 5 aa, Synthetic construct
EVEDE

SEQ ID NO: 254 - protein, 5 aa, Synthetic construct
VEDEA

SEQ ID NO: 255 - protein, 5 aa, Synthetic construct
EDEAL

SEQ ID NO: 256 - protein, 5 aa, Synthetic construct
DEALW

SEQ ID NO: 257 - protein, 5 aa, Synthetic construct
EALWA

SEQ ID NO: 258 - protein, 5 aa, Synthetic construct
ALWAW

SEQ ID NO: 259 - protein, 5 aa, Synthetic construct
LWAWP

SEQ ID NO: 260 - protein, 5 aa, Synthetic construct
SSPGP

SEQ ID NO: 261 - protein, 5 aa, Synthetic construct
SPGPE

SEQ ID NO: 262 - protein, 5 aa, Synthetic construct
PGPEA

SEQ ID NO: 263 - protein, 5 aa, Synthetic construct
GPEAS

SEQ ID NO: 264 - protein, 5 aa, Synthetic construct
ASPLP

SEQ ID NO: 265 - protein, 5 aa, Synthetic construct
SPLPD

SEQ ID NO: 266 - protein, 5 aa, Synthetic construct
PLPDG

SEQ ID NO: 267 - protein, 5 aa, Synthetic construct
LPDGE

SEQ ID NO: 268 - protein, 5 aa, Synthetic construct
PDGES

SEQ ID NO: 269 - protein, 5 aa, Synthetic construct
DGESE

SEQ ID NO: 270 - protein, 5 aa, Synthetic construct
GESEA

SEQ ID NO: 271 - protein, 5 aa, Synthetic construct
ESEAS

SEQ ID NO: 272 - protein, 5 aa, Synthetic construct
SEASR

SEQ ID NO: 273 - protein, 5 aa, Synthetic construct
EASRP

SEQ ID NO: 274 - protein, 5 aa, Synthetic construct
ASRPP

SEQ ID NO: 275 - protein, 5 aa, Synthetic construct
SRPPR

SEQ ID NO: 276 - protein, 5 aa, Synthetic construct
NLASP

SEQ ID NO: 277 - protein, 5 aa, Synthetic construct
LASPS

SEQ ID NO: 278 - protein, 5 aa, Synthetic construct
ASPSP

SEQ ID NO: 279 - protein, 5 aa, Synthetic construct
SPSPS

SEQ ID NO: 280 - protein, 5 aa, Synthetic construct
PSPST

SEQ ID NO: 281 - protein, 5 aa, Synthetic construct
SPSTL

SEQ ID NO: 282 - protein, 5 aa, Synthetic construct
RGESE

SEQ ID NO: 283 - protein, 5 aa, Synthetic construct
GESEE

SEQ ID NO: 284 - protein, 5 aa, Synthetic construct
ESEET

SEQ ID NO: 285 - protein, 5 aa, Synthetic construct
SEETG

SEQ ID NO: 286 - protein, 5 aa, Synthetic construct
EETGS

SEQ ID NO: 287 - protein, 5 aa, Synthetic construct
ETGSS

SEQ ID NO: 288 - protein, 5 aa, Synthetic construct
TGSSE

SEQ ID NO: 289 - protein, 5 aa, Synthetic construct
GSSEG

SEQ ID NO: 290 - protein, 5 aa, Synthetic construct
SSEGA

SEQ ID NO: 291 - protein, 5 aa, Synthetic construct
SEGAP

SEQ ID NO: 292 - protein, 5 aa, Synthetic construct
EGAPS

SEQ ID NO: 293 - protein, 5 aa, Synthetic construct
APSED

SEQ ID NO: 294 - protein, 5 aa, Synthetic construct
PSEDN

SEQ ID NO: 295 - protein, 5 aa, Synthetic construct
SEDNS

SEQ ID NO: 296 - protein, 5 aa, Synthetic construct
EDNSG

SEQ ID NO: 297 - protein, 5 aa, Synthetic construct
DNSGR

SEQ ID NO: 298 - protein, 5 aa, Synthetic construct
NSGRT

SEQ ID NO: 299 - protein, 5 aa, Synthetic construct
SGRTA

SEQ ID NO: 300 - protein, 5 aa, Synthetic construct
GRTAP

SEQ ID NO: 301 - protein, 5 aa, Synthetic construct
RTAPA

SEQ ID NO: 302 - protein, 5 aa, Synthetic construct
TAPAG

SEQ ID NO: 303 - protein, 5 aa, Synthetic construct
APAGT

SEQ ID NO: 304 - protein, 5 aa, Synthetic construct
PAGTS

SEQ ID NO: 305 - protein, 5 aa, Synthetic construct
AGTSV

SEQ ID NO: 306 - protein, 5 aa, Synthetic construct
PAYPA

SEQ ID NO: 307 - protein, 9 aa, Synthetic construct
FLWSDGVPL

SEQ ID NO: 308 - protein, 9 aa, Synthetic construct
FLPLLAALV

SEQ ID NO: 309 - protein, 9 aa, Synthetic construct
FLSGENCVV

SEQ ID NO: 310 - protein, 9 aa, Synthetic construct
ALWAWPSEL

SEQ ID NO: 311 - protein, 9 aa, Synthetic construct
FLGDPPEKL

SEQ ID NO: 312 - protein, 9 aa, Synthetic construct
SLTDVSLAL

SEQ ID NO: 313 - protein, 9 aa, Synthetic construct
VLVARGVRV

SEQ ID NO: 314 - protein, 9 aa, Synthetic construct
GLAQRNLPL

SEQ ID NO: 315 - protein, 9 aa, Synthetic construct
DLYDVYCYA

SEQ ID NO: 316 - protein, 9 aa, Synthetic construct
LLGRWKALL

SEQ ID NO: 317 - protein, 9 aa, Synthetic construct
LLYENWNPG

SEQ ID NO: 318 - protein, 9 aa, Synthetic construct
QLFLPLLAA

SEQ ID NO: 319 - protein, 9 aa, Synthetic construct
RLLGRWKAL

SEQ ID NO: 320 - protein, 9 aa, Synthetic construct
ALPAYPASL

SEQ ID NO: 321 - protein, 9 aa, Synthetic construct
TIPCHVHYL

SEQ ID NO: 322 - protein, 9 aa, Synthetic construct
VLAQAPAAL

SEQ ID NO: 323 - protein, 9 aa, Synthetic construct
QLYAAYLGG

SEQ ID NO: 324 - protein, 9 aa, Synthetic construct
FLFPNQTGF

SEQ ID NO: 325 - protein, 9 aa, Synthetic construct
DLNGELFLG

SEQ ID NO: 326 - protein, 9 aa, Synthetic construct
LWSDGVPLL

SEQ ID NO: 327 - protein, 9 aa, Synthetic construct
MYGAHLASI

SEQ ID NO: 328 - protein, 9 aa, Synthetic construct
FQGACYKHF